(19) United States
(12) Patent Application Publication
(10) Pub. No.: US 2022/0323500 A1
(43) Pub. Date: Oct. 13, 2022

(54) CANCER IMMUNOTHERAPY

(71) Applicant: Achilles Therapeutics UK Limited, London (GB)

(72) Inventors: Katy Newton, Peterborough (GB); Sergio Quezada, London (GB)

(21) Appl. No.: 17/716,737

(22) Filed: Apr. 8, 2022

(30) Foreign Application Priority Data
Apr. 9, 2021 (GB) 2105117.2
Jul. 8, 2021 (GB) 2109886.8

(51) Int. Cl. A61K 35/17; A61P 35/00; A61K 38/20

(52) U.S. Cl. CPC A61K 35/17; A61P 35/00; A61K 38/2013; A61K 2035/124

(57) ABSTRACT

The present invention relates to a method for treating a subject having been diagnosed has having cancer with an immunotherapy is described. The method the following steps: a) providing a sample of a pharmaceutical product comprising T cells; b) analysing the reactivity of the sample T cells to an assay antigen; c) determining that said sample T cells meet a predetermined threshold for reactivity to the assay antigen; and d) if the sample T cells meet the predetermined threshold, administering the pharmaceutical product to the subject, wherein the pharmaceutical product comprise T cells isolated from a tumour sample from the subject, and wherein the T cells are tumour infiltrating lymphocytes (TILs).

### FIELD OF THE INVENTION

The present invention relates to a batch release assay for pharmaceutical products relating to T cell therapies. Said T cells therapies are particularly useful for treating or preventing cancer in a subject.

### BACKGROUND

Cell therapy is a therapy in which viable cells are injected, grafted or implanted into a patient, for example, by grafting stem cells to regenerate diseased tissues or by transplanting T cells capable of fighting cancer cells via cell-mediated immunity in the course of immunotherapy. Cell therapies may be formulated as pharmaceutical products intended for administration to a patient. Pharmaceutical products intended for use in patients are subject to a procedure for the approval and release of the finished product batch. A batch release is a certification of a medicinal product or a drug by an authorized person. The batch release must be performed before the products are administered to patients, either during clinical trials or for approved commercial use.

Batch release assays are intended to predict the activity of a manufactured cellular composition by detecting or measuring one or more biological markers that are linked to one or more physiological properties. It is important to develop batch release assays that reflect the required properties of cellular compositions and provide a reliable measure of production batch-to-batch consistency. Personalised therapies present additional challenges in this regard, as each batch may have originated from a different patient, for example if the patient's own cells have been used to generate the cell therapy. This is the case for T cell therapies in particular, wherein the patient's T cells are isolated, modified and/or expanded, and then returned to the patient.

There is therefore a need in the art for a suitable batch release assay for pharmaceutical products involving T cells.

### SUMMARY OF THE INVENTION

The present inventors have developed a batch release assay suitable for a pharmaceutical product comprising T cells that relies on the ability of the T cells to recognise an antigen.

In one aspect the invention provides a batch release assay for a pharmaceutical product comprising T cells, wherein said assay comprises the step of determining whether said T cells recognise an assay antigen.

The batch release assay may comprise the steps of: a) providing a sample of the pharmaceutical product comprising T cells; b) stimulating the sample T cells with an assay antigen; and c) determining whether the sample T cells recognise said assay antigen.

The sample T cells may be stimulated with said assay antigen in the absence of any other cell types.

The sample T cells may be stimulated with said assay antigen in the absence of antigen presenting cells (APCs).

The sample T cells may present said assay antigen to other T cells (T-to-T assay).

The ability of said T cells to recognise said assay antigen may be assessed by analysis of intracellular or secreted cytokine expression levels, for example expression levels of IFN-γ and/or TNF-α when the T cells are exposed to an assay antigen they recognise. The analysis of expression may be by flow cytometry or immunoassay.

The ability of said T cells to recognise said assay antigen may be assessed by analysis of expression of T cell activation markers in response to stimulation with such antigen. The markers are selected from 4-1BB, CD25, OX40, Ki67, Granzyme B, Perforin, CD107a, LAG-3, PD-1, TIM-3, CTLA4, CD39, Fas, FasL, CD40L, KLRG1, GITR and ICOS. The analysis of expression may be by flow cytometry or immunoassay.

The assay antigen may have been determined to be present in the subject to whom the pharmaceutical product is to be administered.

The assay antigen may be a subject-specific antigen. The assay antigen may be a known tumour antigen, for example a tumour associated antigen (TAA) and/or a tumour specific antigen (TSA).

The assay antigen may be a peptide. In another aspect, the antigen may be encoded by a DNA or RNA molecule.

In one aspect, the assay antigen may be a neoantigen and/or the assay antigen may be a clonal neoantigen.

In one aspect, the pharmaceutical product is for use in the treatment or prevention of cancer in a subject.

Said pharmaceutical product may comprise T cells isolated from a sample, a peripheral blood sample or other tissue sample from the subject. When the T cells are isolated from a tumour sample from the subject, the T cells may be tumour infiltrating lymphocytes (TIL).

In one aspect, said pharmaceutical product comprises T cells that have been expanded in vitro.

In one aspect, the T cells of the batch release assay have been expanded in the presence of an expansion antigen.

The assay antigen may be identified prior to or after T cell expansion.

The assay antigen may be identified as a neoantigen prior to or after T cell expansion.

The assay antigen may be identified as a clonal neoantigen prior to or after T cell expansion.

In one aspect, the same antigen is used both in the step of determining whether said T cells recognise an antigen (assay antigen) and in the T cell expansion (expansion antigen).

In an alternative aspect, a different antigen is used in the step of determining whether said T cells recognise an antigen (assay antigen) to the antigen used in the T cell expansion (expansion antigen).

The invention encompasses a pharmaceutical product comprising T cells that has passed the batch release assay according to the invention, i.e., has been deemed suitable for release for administration to a subject by the batch release assay according to the invention.

Thus, in one aspect, the pharmaceutical product is deemed suitable for release for use in a subject when there is a threshold number of T cells that recognise a particular antigen in said product.

In one aspect the pharmaceutical product may be suitable for release if it comprises at least about from 1×10⁵ reactive cells to 1×10¹³ reactive T cells. Preferably, the pharmaceutical product may be suitable for release if it comprises at least about 1×10⁷ reactive cells.

T cells that recognise a particular assay antigen and/or T cells which are stimulated in response to an assay antigen are considered synonymous with reactive T cells.

In one aspect, the assay antigen may be any suitable antigen for assessing T cell reactivity.

The pharmaceutical product described herein may comprise CAR-T cells or engineered T cells.

In one aspect, the invention provides a method of determining whether a pharmaceutical product comprising T cells is suitable for release for administration to a subject, wherein said method comprises analysing said T cells for reactivity to an assay antigen.

The invention encompasses a pharmaceutical product comprising T cells that has passed the batch release assay according to the invention, i.e., has been deemed suitable for release for administration to a subject by the batch release assay according to the invention.

The method may comprise the steps of: a) providing a sample of a pharmaceutical product comprising T cells; b) analysing the reactivity of the sample T cells to an assay antigen; and c) determining whether said sample T cells meet a predetermined threshold for reactivity to the assay antigen.

In one aspect, step b) of the method may be carried out in the absence of any other cell types.

In one aspect, step b) of the method may be carried out in the absence of antigen presenting cells (APCs).

The T cell reactivity of step b) may be measured by analysis of intracellular or secreted cytokine expression level and/or T cell activation expression level.

The T cell reactivity may be determined by flow cytometry, immunoassay, ELISpot and/or TCR sequencing.

In one aspect, the invention provides a pharmaceutical product comprising T cells which recognise an antigen as determined by the batch release assay. Said pharmaceutical product may be for use in treat and/or preventing cancer. The cancer may be melanoma or non-small cell lung cancer (NSCLC).

In one aspect, the invention provides a method for preventing and/or treating a disease which comprises the following steps: a) providing a sample of a pharmaceutical product comprising T cells; b) analysing the reactivity of the sample T cells to an assay antigen; c) determining that said sample T cells meet a predetermined threshold for reactivity to the assay antigen; and d) if the sample T cells meet the predetermined threshold, administering the pharmaceutical product to the subject.

Step b) of the method may be carried out in the absence of any other cell types. Step b) may be carried out in the absence of antigen presenting cells (APCs).

The disease may be cancer. The cancer may be selected from lung cancer (small cell, non-small cell and mesothelioma), melanoma, bladder cancer, gastric cancer, oesophageal cancer, breast cancer (e.g. triple negative breast cancer), colorectal cancer, cervical cancer, ovarian cancer, endometrial cancer, kidney cancer (renal cell), brain cancer (e.g. gliomas, astrocytomas, glioblastomas), lymphoma, small bowel cancers (duodenal and jejunal), leukaemia, liver cancer (hepatocellular carcinoma), pancreatic cancer, hepatobiliary tumours, germ cell cancers, prostate cancer, Merkel cell carcinoma, head and neck cancers (squamous cell), thyroid cancer, high microsatellite instability (MSI-H), and sarcomas.

### BRIEF DESCRIPTION OF THE DRAWING

The patent or application file contains at least one drawing executed in color. Copies of this patent or patent application publication with color drawing(s) will be provided by the Office upon request and payment of the necessary fee.

FIG. 1 Cell function for patient T-05: Function is measured by cytokine production using flow cytometric analysis. CD3+ T cell cytokine production in response to short peptide pools and CD3+ T cell cytokine production in response to long peptide pools.

FIGS. 2A-2D Tracking cNeT in peripheral circulation allows estimation of the reactive T cell component pre- and post-dosing. RS is the patient rescreening visit, D are visit days post-dosing, W are visit weeks post-dosing. There is detectability of both short (SMP) and long (LMP) peptide reactivity. ELISpot was run in technical triplicates, presented are mean spot forming units (2A). Absolute cell count for B-cells, NK-cells and T-cells were obtained from whole blood TBNK assay and presented as cell count 10⁶/mL blood (2B) and allows for ELISpot mean spot forming unit normalised for the frequency of T-cells per well using TBNK data (2C). 2D shows estimated mean reactive cNeT count/mL in whole blood.

FIG. 3 Upregulation of markers of T cell activation: Marker expression is measured using flow cytometric analysis to determine the frequency of antigen-reactive cells.

FIGS. 4A-4B Intracellular cytokine staining (ICS) assay: The percentage of cytokine positive CD4+ T cells in response to B cells and mDCs pulsed with patient-specific neoantigen peptides pools can measure potency of the product. cNeT were added at ratios of 1:1 (T cell:B cell) or 1:10 (T cell:mDC) and cultured for 16-17 hours.

FIG. 5 Soluble cytokine release: Cell function is measured by cytokine production using cytokine bead array (CBA). Three cytokines were detected in response to peptides, in line with a mitogen positive control.

FIG. 6 Cytokine secretion assay: Supernatants were collected after 44-52 hours and IFNγ measured and quantified by cytometric bead array (CBA) assay using flow cytometry.

FIG. 7 Cytokine secretion assay: Supernatants were collected after 44-52 hours and TNFα measured and quantified by cytometric bead array (CBA) assay using flow cytometry.

FIGS. 8A-8B Peptide reactivity in blood cNeT: (8A) CD3+ T cell cytokine production in response to brefeldin and monensin only (unstimulated) and (8B) CD3+ T cell cytokine production in response to long peptide pools (stimulated).

FIGS. 9A-9B Marker expression in response to peptide stimulation: (9A) Cell surface staining of 4-1BB and CD107a in response to negative control (DMSO) or patient-specific peptides. (9B) Cell surface staining of CD40L and CD25 in response to negative control (DMSO) or patient-specific peptides.

### DETAILED DESCRIPTION OF THE INVENTION

### Batch Release

The present inventors have found that culturing T cells with antigens (such as neoantigens or clonal neoantigens) provides a reliable method of identifying T cells reactive to said antigens which are deemed suitable as a pharmaceutical product for administering to a subject. Notably, said assay is able to identify T cells reactive to multiple different antigens, such as multiple different clonal neoantigens.

Furthermore, the present inventors have surprisingly found that T cells alone, in the absence of any other cell types, such as antigen presenting cells (APCs), can present antigen to other T cells at a level sufficient to enable the detection of antigen-specific reactivity. As such, in one aspect the batch release assay may comprise only T cell subtypes, and is described herein as a T:T cell assay or T-to-T cell assay. This approach removes the need to provide additional cell cultures or generate alternative cell lines such as B cells or dendritic cells (DCs), and provides a more easily validated and scalable assay.

As described above, batches of pharmaceutical products are subject to a batch release assay, which determines whether the batch is suitable for release for administration to a subject.

Suitable batch release processes are usually a regulatory requirement in the field of pharmaceutical/medicinal products. A suitable batch release assay and suitable criteria/cut-off values should be provided for any pharmaceutical products intended for use in a subject.

A batch release assay as described herein may also be referred to as a “potency assay”.

By way of background information, the process of batch release may comprise the following:
(i) The checking of the manufacture and testing of the batch in accordance with defined release procedures.
(ii) The certification of the finished product batch performed by a qualified person (QP) signifying that the batch is in compliance with GMP and the requirements of its Investigational Medicinal Product Dossier (IMPD) or Marketing Authorisation (MA). This represents the quality release of the batch.
(iii) The release of the finished batch of product which should take into account the certification performed by the QP. If this release is performed at a site other than that where certification takes place, then the arrangement should be documented in a written agreement between the sites.

The purpose of controlling batch release is notably to ensure that the batch has been manufactured and checked in accordance with the requirements of its IMPD or MA, the batch has been manufactured and checked in accordance with the principles and guidelines of GMP, and that any other relevant legal requirements are taken into account.

The present invention provides a batch release assay for pharmaceutical products comprising T cells. The present invention requires that T cells are analysed for reactivity to an antigen, for example a specifically-defined or identified antigen. Reactivity to said antigen is used to determine whether the pharmaceutical product or composition is suitable for release.

In one aspect the pharmaceutical product must contain a certain number of T cells or number of T cells reactive to said antigen. In one aspect the pharmaceutical product may be suitable for release if it comprises at least about 1×10⁵, at least about 5×10⁶ reactive cells, preferably at least about 1×10⁷ reactive cells.

In one aspect the invention provides a method for determining whether a pharmaceutical product comprising T cells as described herein is suitable for release for administration to a subject, wherein said method comprises analysing said T cells for reactivity to an antigen as described herein.

In one aspect, use of an antigen as described herein in a batch release assay for a pharmaceutical product comprising T cells, is provided.

### Antigen Reactivity

The batch release assay according to the present invention relies on analysis of whether T cells in the pharmaceutical product recognise an antigen as described herein.

The term “reactive T cell” is used herein to refer to a T cell that recognises an antigen or a T cell that provides a detectable response when stimulated by an antigen. A reactive T cell may be identified by the batch release assay according to the invention.

The term “stimulating” is used herein to refer to exposing a T cell to an antigen, for example by culturing the T cell in the presence of the antigen, suitably under conditions that allow the T cell to provide a detectable response when it recognises or binds to an antigen.

Suitable methods for determining such reactivity will be known in the art.

As described in the present Examples, one way of determining whether T cells recognise (or are reactive to or bind to) an antigen is to analyse the level of cytokine production or secretion by the T cells when the T cells are exposed to said antigen. The level of cytokine production may be analysed by any suitable method, for example flow cytometry. Additionally or alternatively, the level of cytokine production may be analysed by an immunoassay such as, for example, ELISA.

In one aspect the T cells may be cultured in the presence of protein transport inhibitors, such as Brefeldin A and Monensin, which prevent release of cytokines from the cell prior to the analysis of cytokine production. Other suitable protein transport inhibitors are envisaged.

In one aspect the cytokine(s) may be IFN-γ and/or TNF-α.

In one aspect, changes in the expression levels of surface markers or markers of T cell activation may be analysed in response to exposure to said antigen(s). These may include, for example, 4-1BB, CD25, OX40, Ki67, Granzyme B, Perforin, CD107a, LAG-3, PD-1, TIM-3, CTLA4, CD39, Fas, FasL, CD40L, KLRG1, GITR and ICOS. Markers may be used for determining the frequency of antigen-reactive cells in the product.

Similarly, surface markers or markers of T cell activation may be analysed by any suitable method, for example flow cytometry. The antigen may be as described herein. In one aspect the antigen is a subject-specific antigen. In an alternative aspect the antigen is a known tumour antigen.

The antigen may be disease-specific, for example cancer-specific, or associated with a disease, such as cancer.

In one aspect the antigen has been identified prior to T cell expansion as being present in the subject's tumour.

In another aspect, the antigen has been identified after T cell expansion as being present in the subject's tumour.

The pharmaceutical product comprising T cells may be a personalised T cell therapy specific for a particular subject.

The batch release assay may rely on analysing reactivity to a specific antigen, rather than analysing merely T cell activity in general (activity that is not antigen-specific) or analysing reactivity to a general antigen that is known to stimulate T cells non-specifically.

Examples of general antigens that are known to non-specifically stimulate T cells include the Staphylococcus Entertoxin B (SEB) superantigen, PMA or ionomycin. Other examples may be use of an anti-CD3 antibody such as OKT3, phytohemagglutinin (PHA) or Concanavalin A (ConA).

In one aspect of the batch release assay according to the invention more than one antigen may be used in the assay, defined herein as an assay antigen. For example, a pool of assay antigens (such as peptides) may be used. The same pool of peptides may have been used to expand the T cells prior to batch release, defined herein as expansion antigens. In this case, the assay antigen and the expansion antigen are the same antigens.

Alternatively, a pool of different peptides may have been used to expand the T cells prior to batch release. In this case, the assay antigen and the expansion antigen are different antigens.

In one aspect, the batch release assay relies on T cells to present the antigen to other T cells. This is referred to herein as a T:T assay or a T-to-T cell assay. Thus in one aspect of the invention, the sample T cells are stimulated with said assay antigen in the absence of any other cell types.

In one aspect, the T cells are stimulated with said assay antigen in the absence of antigen presenting cells (APCs).

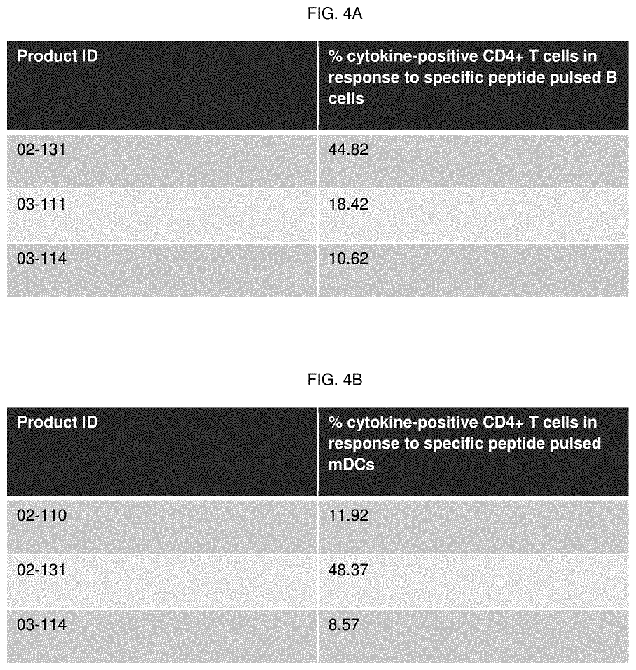

In one aspect, the batch release assay described herein may comprise a combination of T cells and non-T cells to present antigen, such as for example B cells (FIGS. 4A-4B).

An antigen-presenting cell (APC) is a cell that displays antigen complexed with major histocompatibility complexes (MHCs) on their surfaces; this process is known as antigen presentation. T cells may recognize these complexes using their T cell receptors (TCRs).

An example of an APC is a dendritic cell.

In one aspect, the pool of peptides may be a masterpool of long peptides and/or a masterpool of short peptides. A pool of long peptides and a pool of short peptides may generally correspond to the CD4+ and CD8+ T cell subsets respectively. The peptides are presented to the T cells in the context of MHC class I (to CD8) and II (to CD4). Peptides which bind to MHC class II molecules are typically between 15 and 24 amino acids in length, and may be as long as 40 amino acids. Thus, a pool of long peptides may comprise peptides between 15 and 40 amino acids in length, for example 27 to 31 amino acids long. Peptides which bind to MHC class I molecules are typically between 7 and 13 amino acids in length. Thus, a pool of short peptides may comprise peptides between 7 and 13 amino acids in length.

### Peptide Selection

As mentioned above, the batch release assay described herein comprises the step of determining whether said T cells recognise an antigen. In further embodiments the T cells are expanded in the presence of an antigen (e.g. prior to batch release). The antigen may be a peptide. Thus, a peptide or pool or peptides may be used for T cell expansion and/or determination of T cell reactivity to one or more antigens. The peptides or pool of peptides may correspond to the one or more antigens, i.e. they may each encode at least part of the amino acid sequence of a peptide or protein that is likely to be antigenic, such as a tumour-specific antigen (TSA), tumour-associated antigen (TAA), neoantigen (such as a clonal neoantigen).

Where at least part of the amino acid sequence of the peptide or protein that is likely to be antigenic is a tumour antigen (e.g. TSA or TAA), it may include any one or more of the following: CEA, immature laminin receptor, TAG-72, HPV E6 and E7, BING-4, calcium-activated chloride channel 2, cyclin-B1, 9D7, Ep-CAM, EphA3, Her2/neu, telomerase, mesothelin, SAP-1, survivin, BAGE family, CAGE family, GAGE family, MAGE family, SAGE family, XAGE family, NY-ESO-1/LAGE-1, PRAME, SSX-2, Melan-A/MART-1, gp100/pmel17, tyrosinase, TRP-1/-2, MC1R, prostate-specific antigen, beta-catenin, BRCA1/2, CDK4, CML66, fibronectin, MART-2, p53, ras, TGF-betaRII and MUC1.

Tumour antigens may also include the following: 707-AP=707 alanine proline, AFP=alpha (α)-fetoprotein, ART-4=adenocarcinoma antigen recognized by T cells 4, BAGE=B antigen; β-catenin/m, β-catenin/mutated, Bcr-abl=breakpoint cluster region-Abelson, CAMEL=CTL-recognized antigen on melanoma, CAP-1=carcinoembryonic antigen peptide-1, CASP-8=caspase-8, CDC27m=cell-division-cycle 27 mutated, CDK4/m=cycline-dependent kinase 4 mutated, CEA=carcinoembryonic antigen, CT=cancer/testis (antigen), Cyp-B=cyclophilin B, DAM=differentiation antigen melanoma (the epitopes of DAM-6 and DAM-10 are equivalent, but the gene sequences are different. DAM-6 is also called MAGE-B2 and DAM-10 is also called MAGE-B1), ELF2M=elongation factor 2 mutated, ETV6-AML1=Etsvariant gene 6/acute myeloid leukemia 1 gene ETS, G250=glycoprotein 250, GAGE=G antigen, GnT-V=N-acetylglucosaminyltransferase V, Gp100=glycoprotein 100 kD, HAGE=helicose antigen, HER-2/neu=human epidermal receptor-2/neurological, HLA-A*0201-R1701=arginine (R) to isoleucine (1) exchange at residue 170 of the α-helix of the α2-domain in the HLA-A2 gene, HPV-E7=human papilloma virus E7, HSP70-2M=heat shock protein 70-2 mutated, HST-2=human signet ring tumor-2, hTERT or hTRT=human telomerase reverse transcriptase, iCE=intestinal carboxylesterase, KIAA0205=name of the gene as it appears in databases, LAGE=L antigen, LDLR/FUT=low density lipid receptor/GDP-L-fucose: β-D-galactosidase 2-α-L-fucosyltransferase, MAGE=melanoma antigen, MART-1/Melan-A=melanomaantigen recognized by T cells-1/Melanoma antigen A, MC1R=melanocortin 1 receptor, Myosin/m=myosin mutated üMUC1=mucin 1, MUM-1, -2, -3=melanomaubiquitous mutated 1, 2, 3, NA88-A=NA cDNA clone of patient M88, NY-ESO-1=New York—esophageous 1, P15=protein 15, p190 minor bcr-abl=protein of 190 3KD bcr-abl, Pml/RARa=promyelocytic leukaemia/retinoic acid receptor a, PRAME=preferentially expressed antigen of melanoma, PSA=prostate-specific antigen, PSM=prostate-specific membrane antigen, RAGE=renal antigen, RU1 or RU2=renalubiquitous 1 or 2, SAGE=sarcoma antigen, SART-1 or SART-3=squamous antigenrejecting tumor 1 or 3, TEL/AML1=translocation Ets-family leukemia/acute myeloidleukemia 1, TPI/m=triosephosphate isomerase mutated, TRP-1=tyrosinase relatedprotein 1, or gp75, TRP-2=tyrosinase related protein 2, TRP-2/INT2=TRP-2/intron2, WT1=Wilms' tumor gene.

For each set of one or more antigens, a set of candidate peptides (whether long or short) may be selected for inclusion in a pool of peptides to be used for T cell expansion and/or batch release based on one or more criteria. In other words, a pool of peptides may be designed for a set of antigens (e.g. a set of one or more proteins/polypeptides that are TSAs, TAAs, neoantigens and/or clonal neoantigens) by providing a set of candidate peptides that each encode at least a part of the amino acid sequence of the antigen, and selecting one or more peptides for inclusion in the pool of peptides based on one or more criteria.

In one aspect, the set of candidate peptides may comprise all peptides of a predetermined length or set of lengths that include one or more predetermined positions of the one or more antigens. For example, the set of candidate peptides may comprise all peptides of predetermined length(s) that include a predetermined position of each of one or more antigens (such as e.g. a position that was determined to be mutated in tumour cells compared to normal cells). As a specific example, the set of candidate peptides may comprise all 8-mer, 9-mer, 10-mer and 11-mer peptides encoding a point mutation (such as a mutation present in tumour cells but not in normal cells) at each possible position from the first to the last position in the peptides, as described in Marty et al. (Cell 2017 Nov. 30; 171(6):1272-1283).

In another aspect, the set of candidate peptides may comprise a subset of all peptides of a predetermined length or set of lengths that include one or more predetermined positions of the one or more antigens. For example, the set of candidate peptides may comprise peptides of a predetermined length including a predetermined position of each of one or more antigens (such as e.g. a position that was determined to be mutated in tumour cells compared to normal cells) at a predetermined location in the peptide. As a specific example, the set of candidate peptides may comprise 25-mer peptides each encoding a point mutation (e.g. a mutation present in tumour cells but not in normal cells) flanked on both sides with 12 wild type (normal) amino acids, as described in Leko et al. (Journal of Immunology, 2019, 202: 000-000). As another specific example, the set of candidate peptides may comprise 21-mer peptides each encoding a tumour-specific variant amino acid placed as near as possible to the centre of the 21-mer, as described in Liu & Mardis (Cell 168, Feb. 9, 2017).

In one aspect of the batch release assay described herein, the pool of peptides are selected from a set of candidate peptides using an in silico screening approach. This approach may, for example, identify candidate peptides predicted to have high affinity to autologous HLA molecules and/or have a high likelihood of being presented by autologous HLA molecules. For example, a suitable peptide may comprise a tumour specific neo-epitope having a tumour specific mutation, where the mutations are only present in the genome of cancer cells, for example as determined by whole genome or whole exome nucleic acid sequencing of the tumour and normal tissue. The mutant peptide may comprise a tumour specific neoepitope which binds to class I HLA protein with greater affinity than the corresponding WT peptide, and has a IC50 of less than 500 nM. (WO2011/028531).

In embodiments, the one or more criteria used to select candidate peptides for inclusion in the pool of peptides comprise one or more criteria selected from: a criterion based on the predicted binding affinity of the candidate peptides to one or more MHC class I and/or class II molecules, a criterion based on the predicted likelihood of presentation of the candidate peptides by one or more MHC class I and/or class II molecules, an expression based criterion that excludes peptides associated with genes for which there is no evidence of expression in a relevant sample or population, a criterion based on the variant allele fraction of a variant present in the peptide in one or more samples from the patient, a criterion based on the predicted binding affinity of wild type counterparts of the candidate peptides to one or more MHC class I and/or class II molecules, a criterion based on the sequence coverage for a genomic coordinate comprising the coding sequence for the peptide in one or more samples from the patient, a criterion based on the predicted binding affinity of peptides comprising a mutation present in the candidate peptides to one or more MHC class I and/or class II molecules, and a criterion based on the recognition, by T cells or tumour infiltrating lymphocytes, of peptides comprising mutations present in the candidate peptides expressed by an engineered cell line also expressing one or more MHC class I and/or class II molecules. The one or more MHC class I and/or class II molecules may be selected based on the HLA haplotype of a patient. The one or more MHC class I and/or class II molecules may include all of the alleles in a patient's HLA haplotype.

Any method known in the art for determining the HLA haplotype of a patient may be used. The HLA haplotype of a patient may be derived from sequencing data (such as e.g. whole exome sequencing data) using methods known in the art, such as e.g. Polysolver (Shukla et al., 2015), HLAMiner (Warren et al., 2012) and Optitype (Szolek et al., 2014).

In silico methods for prediction of MHC class 1 binding affinity and/or presentation are known in the art and include netMHC (Lundegaard et al. Bioinformatics, Volume 24, Issue 11, 1 Jun. 2008, Pages 1397-1398, Nielsen et al. Protein Sci., (2003) 12:1007-17), netMHCstabpan (Rasmussen et al., Journal of Immunology, 2016), NetMHPCpan (Jurtz et al., The Journal of Immunology (2017) ji1700893), and MHCflurry (O'Donnell et al., Cell Systems, 7(1), 25 Jul. 2018, 129-132.e4). In silico methods for prediction of MHC class II binding affinity and/or presentation are known in the art and include NetMHCIIpan-4.0 (Reynisson et al., J Proteome Res. 2020 Jun. 5; 19(6):2304-2315), BERTMHC (bioarxiv 2020, https COLON SLASH SLASH doi.org/10.1101/2020.11.24.396101), amongst others.

Further, in silico methods for prediction of MHC presentation likelihood may reflect at least in part the stability of binding between the candidate peptides and the one or more MHC molecules. Methods for prediction of MHC class 1 binding affinity and/or presentation which take into account the stability of binding include NetMHCstab (Jorgensen et al., Immunology 2014 January; 141(1): 18-26) and the method described in Jappe et al. (Nature Communications 11, Article number: 6305 (2020)).

A criterion based on MHC binding affinity and/or presentation for the wild type counterparts of the candidate peptides may include the exclusion of candidate peptides with a difference between predicted MHC binding affinity and/or presentation likelihood for the peptide and wild type counterpart below (respective) thresholds, or the inclusion of the top x candidate peptides (where x can be e.g. 1, 2, 5, 10, 15, 20, 50, 100, 150, etc.) with the highest difference in predicted MHC binding affinity and/or presentation likelihood between the peptide and the wild type counterpart (optionally the top x candidate peptides that also satisfy one or more of the additional criteria mentioned herein).

A criterion based on MHC binding affinity and/or presentation the candidate peptides may include the exclusion of candidate peptides with a predicted MHC binding affinity and/or presentation likelihood below (respective) thresholds, or the inclusion of the top x candidate peptides (where x can be e.g. 1, 2, 5, 10, 15, 20, 50, 100, 150, etc.) with the highest predicted MHC binding affinity and/or presentation likelihood (optionally the top x candidate peptides that also satisfy one or more of the additional criteria mentioned herein).

An expression-based criterion that excludes peptides associated with genes for which there is no evidence of expression in a relevant sample or population may be based on RNA expression data from a sample from a patient or a relevant population. For example, peptides may be excluded if they are associated with genes that are not expressed in a sample from the patient and/or that are not expressed in a population of samples from a particular tissue (e.g. the tissue in which a patient's cancer is expected to be located or from which a patient's cancer is expected to be originating) or a particular group of subjects (e.g. a group of subjects having a particular type of cancer).

A criterion based on the variant allele fraction of a variant present in the peptide may exclude peptides comprising a somatic variant that is present in a tumour sample of the patient (or estimated to be present in the tumour cells of a patient) at a variant allele fraction below a threshold. Such a criterion may be based at least in part on the number of reads in DNA sequencing data from a tumour sample from the patient that support the variant. A criterion based on variant allele fraction of a variant present in the peptide may exclude peptides that have an estimated likelihood of being clonal and/or an estimate cancer cell fraction that is below a (respective) threshold. A criterion based on variant allele fraction of a variant present in the peptide may include the top x candidate peptides (where x can be e.g. 1, 2, 5, 10, 15, 20, 50, 100, 150, etc.) with the highest predicted likelihood of being clonal and/or the highest predicted cancer cell fraction (optionally the top x candidate peptides that also satisfy one or more of the additional criteria mentioned herein).

A criterion based on the sequence coverage for a genomic coordinate comprising the coding sequence for the peptide in one or more samples from the patient may exclude peptides comprising a somatic variant at a genomic location for which the coverage in DNA sequencing data from a tumour sample and/or a normal sample is below a predetermined threshold.

A criterion based on the predicted binding affinity of a peptide comprising a mutation present in the candidate peptides to one or more MHC class I and/or class II molecules may exclude peptides comprising a mutation for which a score estimating the qualitative likelihood of MHC presentation is below a threshold, or the inclusion of the candidate peptides comprising the top x mutations (where x can be e.g. 1, 2, 5, 10, 15, 20, 50, 100, 150, etc.) with the highest score estimating the qualitative likelihood of MHC presentation (optionally the top x candidate peptides that also satisfy one or more of the additional criteria mentioned herein). A score estimating the qualitative likelihood of MHC presentation for a mutant may be obtained by obtaining the predicted MHC binding affinities across all possible peptides of a predetermined length or range of lengths that include the mutant, for a plurality of candidate peptides, and assigning a mutation-level score for each mutation in the set of candidate peptides. The mutation-level score may be the best rank of any peptide comprising the mutation, in a ranked list of the obtained predicted MHC binding affinities, for example as described in Marty et al., Cell 2017 Nov. 30; 171(6): 1272-1283.

A criterion based on the recognition of peptides comprising mutations present in the candidate peptides expressed by an engineered cell line also expressing one or more MHC class I and/or class II molecules by T cells or tumour infiltrating lymphocytes may comprise the selection of peptides that comprise mutants that, when expressed by said cell line, cause the T cells/TIL to produce a cytokine, such as IL-2 or IFNγ (or to produce a cytokine such as IFNγ in amounts above a predetermined threshold). The engineered cell line may have been engineered to express a tandem minigene library encoding polypeptides comprising the mutations present in the candidate peptides, such as e.g. as described in Lu et al. (Clinical Cancer Res. 2014 Jul. 1; 20(13):3401-3410.

The engineered cell line may be an antigen presenting cell. The engineered cell line may have been further engineered to express an anti-cytokine antibody. The anti-cytokine antibody may be expressed on the surface of the engineered cells and may enable the identification of engineered cells bound to the cytokine, wherein binding of the cytokine to the engineered cell is indicative of an interaction between a T cell receptor expressed by the T cells co-cultured with the engineered cell, and the epitope presented by the engineered cell (i.e. the candidate peptide or a processed version of a polypeptide corresponding to the candidate peptide). For example, a protocol for screening candidate antigens using engineered antigen-presenting cells as described in Lee & Meyerson (Sci Immunol. 2021 Jan. 22; 6(55):eabf4001) may be used.

### T Cell Therapy

The present invention relates to pharmaceutical products comprising T cells. Such pharmaceutical products as referred to herein may be T cell therapies.

In one aspect the T cells may be the major or only active agent in the pharmaceutical product.

T cells therapies may be used as immunotherapy.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy can include adoptive T cell therapy, autologous T cell therapy, tumour-infiltrating lymphocyte (TIL) therapy, engineered T cell therapy, chimeric antigen receptor (CAR) T cell therapy, engineered TCR T cell therapy and allogeneic T cell transplantation. Examples of T cell therapies are described in International Publication Nos, WO2018/002358, WO2013/088114, WO2015/077607, WO2015/143328, WO2017/049166 and WO2011/140170.

The T cells of the immunotherapy may originate from any source known in the art. For example, T cells may be differentiated in vitro from a hematopoietic stem cell population, or T cells can be obtained from a subject. T cells may be obtained from, e.g., peripheral blood mononuclear cells, bone marrow, lymph node tissue, cord blood, thymus tissue, tissue from a site of infection, ascites, pleural effusion, spleen tissue, and tumours. In addition, the T cells may be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety.

In one aspect of the invention as described herein, a single dose of T cell therapy is administered to the patient. In one aspect a single dose of T cell therapy is administered to the patient on day 0 only. In other aspects of the invention, multiple doses of T cell therapy are administered to the patient starting from day 0. For example, the number of doses of T cell therapy may be 2, 3, 4, 5, 6, 7, 8, 9, 10 or more than 10 doses.

Dosing may be once, twice, three times, four times, five times, six times, or more than six times per year. Alternatively, dosing may be once, twice, three times, four times, five times, six times, or more than six times per month. In a further aspect dosing may be once, twice, three times, four times, five times, six times, or more than six times every two weeks. In yet a further aspect dosing may be once, twice, three times, four times, five times, six times, or more than six times per week, for example once a week, or once every other day.

Administration of the T cell therapy may continue as long as necessary.

In one aspect the T cell therapy may comprise CD8+ T cells, CD4+ T cells or CD8+ and CD4+ T cells.

The T cell therapy as described herein may be used in vitro, ex vivo or in vivo, for example either for in situ treatment or for ex vivo treatment followed by the administration of the treated cells to the body.

In certain aspects according to the invention as described herein the T cell therapy is reinfused into a subject, for example following T cell isolation and expansion as described herein. Suitable methods for generating, selecting, expanding and reinfusing T cells are known in the art.

The T cell therapy may be administered to a subject at a suitable dose. The dosage regimen may be determined by the attending physician and clinical factors. It is accepted in the art that dosages for any one patient depend upon many factors, including the patient's size, body surface area, age, the particular compound to be administered, sex, time and route of administration, general health, and other drugs being administered concurrently.

The T cell therapy may involve the transfer of a given number of T cells as described herein to a patient, for example TILs or CAR-T cells. The therapeutically effective amount of T cells or antigen reactive cells may be at least about 103 cells, at least about 104 cells, at least about 105 cells, at least about 106 cells, at least about 107 cells, at least about 108 cells, at least about 109 cells, at least about 1010 cells, at least about 1011 cells, at least about 1012 or at least about 1013 cells.

Other suitable doses of T cells may be as described in, for example, WO 2016/191755, WO2019/112932, WO2018/226714, WO2018/182817, WO2018/129332, WO2018/129336, WO2018/094167, WO2018/081789 and WO2018/081473.

### Tumour-Infiltrating Lymphocyte (TIL) Therapy

In one aspect of the invention the T cell therapy uses TILs.

Tumour-infiltrating lymphocyte (TIL) immunotherapy is a type of adoptive T cell therapy wherein T cells that have infiltrated tumour tissue are isolated, enriched in vitro and administered to a patient. Generation of TIL cultures may be performed by first culturing resected tumour fragments or tumour single-cell suspensions in medium containing IL-2. This initial pre-expansion may be followed by a rapid expansion protocol (REP) involving the activation of TILs using an anti-CD3 monoclonal antibody in the presence of irradiated peripheral blood mononuclear cells (PBMC) and IL-2. Examples of TIL therapies and expansion protocols are described in International Patent Publication No.s WO2018/081473, WO2018/081789, WO2018/094167, WO2018/129336, WO2018/129332, WO2018/182817, WO2018/226714, WO2019/100023, WO2019/112932 and US granted patent No.s U.S. Pat. Nos. 8,383,099 and 9,074,185.

### Engineered T Cell Therapy

In one aspect of the invention the T cell therapy uses engineered T cells. The T cells are isolated from the patient (e.g. from a blood sample) and are modified, for example to express a chimeric antigen receptor (CAR) or a TCR receptor that binds to a target antigen.

CARs are proteins which, in their usual format, graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals.

The most common form of these molecules uses single-chain variable fragments (scFv) derived from monoclonal antibodies to recognize a target antigen. The scFv is fused via a spacer and a transmembrane domain to a signalling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

Affinity-enhanced TCRs are generated by identifying a T cell clone from which the TCR a and β chains with the desired target specificity are cloned. The candidate TCR then undergoes PCR directed mutagenesis at the complimentary determining regions of the α and β chains. The mutations in each CDR region are screened to select for mutants with enhanced affinity over the native TCR. Once complete, lead candidates are cloned into vectors to allow functional testing in T cells expressing the affinity-enhanced TCR.

T cells may bear high affinity TCRs, and hence affinity enhancement may not be necessary. High affinity TCRs may be isolated from T cells from a subject and may not require affinity enhancement.

Identified TCRs and/or CARs may be expressed in autologous T cells from a subject using methods which are known in the art, for example by introducing DNA or RNA coding for the TCR or CAR by one of many means including transduction with a viral vector, transfection with DNA or RNA.

### Antigens

In one aspect of the invention the T cell therapy comprises T cells which target cancer-associated or tumour-specific antigens.

Tumour antigens include the following: CEA, immature laminin receptor, TAG-72, HPV E6 and E7, BING-4, calcium-activated chloride channel 2, cyclin-B1, 9D7, Ep-CAM, EphA3, Her2/neu, telomerase, mesothelin, SAP-1, survivin, BAGE family, CAGE family, GAGE family, MAGE family, SAGE family, XAGE family, NY-ESO-1/LAGE-1, PRAME, SSX-2, Melan-A/MART-1, gp100/pmel17, tyrosinase, TRP-1/-2, MC1R, prostate-specific antigen, prostate-specific membrane antigen, beta-catenin, BRCA1/2, CDK4, CML66, fibronectin, MART-2, p53, ras, TGF-betaRII and MUC1.

Tumour antigens may also include the following: 707-AP=707 alanine proline, AFP=alpha (α)-fetoprotein, ART-4=adenocarcinoma antigen recognized by T cells 4, BAGE=B antigen; β-catenin/m, β-catenin/mutated, Bcr-abl=breakpoint cluster region-Abelson, CAMEL=CTL-recognized antigen on melanoma, CAP-1=carcinoembryonic antigen peptide-1, CASP-8=caspase-8, CDC27m=cell-division-cycle 27 mutated, CDK4/m=cycline-dependent kinase 4 mutated, CEA=carcinoembryonic antigen, CT=cancer/testis (antigen), Cyp-B=cyclophilin B, DAM=differentiation antigen melanoma (the epitopes of DAM-6 and DAM-10 are equivalent, but the gene sequences are different. DAM-6 is also called MAGE-B2 and DAM-10 is also called MAGE-B1), ELF2M=elongation factor 2 mutated, ETV6-AML1=Etsvariant gene 6/acute myeloid leukemia 1 gene ETS, G250=glycoprotein 250, GAGE=G antigen, GnT-V=N-acetylglucosaminyltransferase V, Gp100=glycoprotein 100 kD, HAGE=helicose antigen, HER-2/neu=human epidermal receptor-2/neurological, HLA-A*0201-R1701=arginine (R) to isoleucine (1) exchange at residue 170 of the α-helix of the α2-domain in the HLA-A2 gene, HPV-E7=human papilloma virus E7, HSP70-2M=heat shock protein 70-2 mutated, HST-2=human signet ring tumor-2, hTERT or hTRT=human telomerase reverse transcriptase, iCE=intestinal carboxylesterase, KIAA0205=name of the gene as it appears in databases, LAGE=L antigen, LDLR/FUT=low density lipid receptor/GDP-L-fucose: β-D-galactosidase 2-α-L-fucosyltransferase, MAGE=melanoma antigen, MART-1/Melan-A=melanomaantigen recognized by T cells-1/Melanoma antigen A, MC1R=melanocortin 1 receptor, Myosin/m=myosin mutated üMUC1=mucin 1, MUM-1, -2, -3=melanomaubiquitous mutated 1, 2, 3, NA88-A=NA cDNA clone of patient M88, NY-ESO-1=New York—esophageous 1, P15=protein 15, p190 minor bcr-abl=protein of 190 3KD bcr-abl, Pml/RARa=promyelocytic leukaemia/retinoic acid receptor a, PRAME=preferentially expressed antigen of melanoma, PSA=prostate-specific antigen, PSM=prostate-specific membrane antigen, RAGE=renal antigen, RU1 or RU2=renalubiquitous 1 or 2, SAGE=sarcoma antigen, SART-1 or SART-3=squamous antigenrejecting tumor 1 or 3, TEL/AML1=translocation Ets-family leukemia/acute myeloidleukemia 1, TPI/m=triosephosphate isomerase mutated, TRP-1=tyrosinase relatedprotein 1, or gp75, TRP-2=tyrosinase related protein 2, TRP-2/INT2=TRP-2/intron2, WT1=Wilms' tumor gene.

In one aspect the antigen may be in the form of nucleic acid, for example DNA and/or RNA molecule. In other words, the step of determining whether a T cell recognises a neoantigen may comprise using a DNA and/or RNA molecule that encodes the antigen. In embodiments, the DNA and/or RNA molecule may encode a plurality of antigens. In embodiments, the DNA and/or RNA molecule may be a minigene or tandem minigene. For example, one or more tandem minigenes encoding one or more antigens may be transfected in antigen presenting cells, as described e.g. in Lu et al. (Molecular Therapy, Vol. 26 No. 2 Feb. 2018) or Leko et al. (J Immunol, 2019, 202:000-000).

### Neoantigens

Many antigens expressed by cancer cells are self-antigens, present both in healthy tissue as well as in cancer cells. Tumour associated antigens (TAA) are those preferentially expressed in tumor cells (or expressed at very high level in tumour cells) whilst present at lower levels or only in some healthy tissues or in specific differentiation stages of healthy tissues. These TAAs are difficult to target with cellular immunotherapy because they require overcoming both central tolerance (whereby autoreactive T cells are deleted in the thymus during development) and peripheral tolerance (whereby mature T cells are suppressed by regulatory mechanisms). In cases in which central and peripheral tolerance are overcome cellular therapies targeting TAA could promote severe on target, off tumour toxicity upon targeting of the TAA expressed in healthy tissue.

These tolerance mechanisms may be abrogated by the targeting of neoantigens. Neoantigens are antigens that are present in cancer cells but not in healthy cells. Because neoantigens are not recognised as ‘self-antigens’, T cells which are capable of targeting neoantigens are not subject to central and peripheral tolerance mechanisms to the same extent as T cells which recognise self-antigens. Furthermore, because tumour neoantigens are expressed only by the tumour cells and not healthy tissue, T cells therapies against neoantigens will not induce specific destruction of healthy, non-tumour tissues.

T cells that specifically recognise neoantigens will only attack cancer cells and not harm normal healthy tissues. Therefore, cellular immunotherapies comprising neoantigen-specific T cells are considered an ideal candidate to treat cancer patients. For this reason, an assay which measures T cell reactivity to neoantigens is considered more relevant and desirable than an assay that measures T cell reactivity to any other antigens (e.g., self-antigens such as TAAs).

Accordingly, in one aspect of the invention the antigen may be a neoantigen.

A “neoantigen” is a tumour-specific antigen which arises as a consequence of a mutation within a cancer cell. Thus, a neoantigen is not expressed (or expressed at a significantly lower level) by healthy (i.e. non-tumour) cells in a subject.

A neoantigen may be processed to generate distinct peptides which can be recognised by T cells when presented in the context of MHC molecules. As described herein, neoantigens may be used as the basis for cancer immunotherapies. References herein to “neoantigens” are intended to include also peptides derived from neoantigens. The term “neoantigen” as used herein is intended to encompass any part of a neoantigen that is immunogenic. An “antigenic” molecule as referred to herein is a molecule which itself, or a part thereof, is capable of stimulating an immune response, when presented to the immune system or immune cells in an appropriate manner. The binding of a neoantigen to a particular MHC molecule (encoded by a particular HLA allele) may be predicted using methods which are known in the art. Examples of methods for predicting MHC binding include those described by Lundegaard et al., O'Donnel et al., and Bullik-Sullivan et al. For example, MHC binding of neoantigens may be predicted using the netMHC-3 (Lundegaard et al.) and netMHCpan4 (Jurtz et al.) algorithms. A neoantigen that has been predicted to bind to a particular MHC molecule is thereby predicted to be presented by said MHC molecule on the cell surface.

The neoantigen described herein may be caused by any non-silent mutation which alters a protein when expressed by a cancer cell compared to the non-mutated protein expressed by a wild-type, healthy cell. In other words, the mutation results in the expression of an amino acid sequence that is not expressed, or expressed at a very low level in a wild-type, healthy cell. For example, the mutation may occur in the coding sequence of a protein, thus altering the amino acid sequence of the resulting protein. This may be referred to as a “coding mutation”. As another example, the mutation may occur in a splice site, thus resulting in the production of a protein that contains a set of exons that is different or less common in the wild type protein. As a further example, the mutated protein may be caused by a translocation or fusion.

A “mutation” refers to a difference in a nucleotide sequence (e.g. DNA or RNA) in a tumour cell compared to a healthy cell from the same individual. The difference in the nucleotide sequence can result in the expression of a protein which is not expressed by a healthy cell from the same individual. For example, the mutation may be one or more of a single nucleotide variant (SNV), a multiple nucleotide variant (MNV), a deletion mutation, an insertion mutation, an indel mutation, a frameshift mutation, a translocation, a missense mutation, a splice site mutation, a fusion, or any other change in the genetic material of a tumour cell.

An “indel mutation” refers to an insertion and/or deletion of bases in a nucleotide sequence (e.g. DNA or RNA) of an organism. Typically, the indel mutation occurs in the DNA, preferably the genomic DNA, of an organism. In embodiments, the indel may be from 1 to 100 bases, for example 1 to 90, 1 to 50, 1 to 23 or 1 to 10 bases. An indel mutation may be a frameshift indel mutation. A frameshift indel mutation is an insertion or deletion of one or more nucleotides that causes a change in the reading frame of the nucleotide sequence. Such frameshift indel mutations may generate a novel open-reading frame which is typically highly distinct from the polypeptide encoded by the non-mutated DNA/RNA in a corresponding healthy cell in the subject.

The mutations may be identified by exome sequencing, RNA-seq, whole genome sequencing and/or targeted gene panel sequencing and/or routine Sanger sequencing of single genes. Suitable methods are known in the art. Descriptions of exome sequencing and RNA-seq are provided by Boa et al. (Cancer Informatics. 2014; 13(Suppl 2):67-82.) and Ares et al. (Cold Spring Harb Protoc. 2014 Nov. 3; 2014(11):1139-48); respectively. Descriptions of targeted gene panel sequencing can be found in, for example, Kammermeier et al. (J Med Genet. 2014 November; 51(11):748-55) and Yap K L et al. (Clin Cancer Res. 2014. 20:6605). See also Meyerson et al., Nat. Rev. Genetics, 2010 and Mardis, Annu Rev Anal Chem, 2013. Targeted gene sequencing panels are also commercially available (e.g. as summarised by Biocompare ((http://www.biocompare.com/Editorial-Articles/161194-Build-Your-Own-Gene-Panels-with-These-Custom-NGS-Targeting-Tools/)).

Sequence alignment to identify nucleotide differences (e.g. SNVs) in DNA and/or RNA from a tumour sample compared to DNA and/or RNA from a non-tumour sample may be performed using methods which are known in the art. For example, nucleotide differences compared to a reference sample may be performed using the method described by Koboldt et al. (Genome Res. 2012; 22: 568-576). The reference sample may be the germline DNA and/or RNA sequence.

### Clonal Neoantigens

Different regions of tumours may be morphologically distinct. In addition, intratumour mutational heterogeneity may occur and can be associated with differences in tumour prognosis and the potential ability of tumour cells to escape immune therapies targeting mutations which are not present in all or most tumour cells.

Intratumour heterogeneity can cause variation between the neoantigens expressed in different regions of a tumour and between different cells in a tumour. In particular, certain neoantigens are expressed in all regions and essentially all cells of the tumour whilst other neoantigens are only expressed in a subset of tumour regions and cells.

The assay described herein provides a method for measuring T cell reactivity to clonal neoantigens. The inventors find this assay to be an improvement over measuring reactivity to other antigens because the assay described herein will determine T cells which could potentially target substantially every cancer cell in different regions of the tumour. This therefore has an increased chance of effectively eliminating all tumour cells and tissues and/or of reducing the likelihood of relapse. The effect, measured by the assay described herein, is improved further if the T cells target multiple clonal antigens.

The inventors hypothesise that a T cell product that is clonal neoantigen reactive is most advantageous as such antigens are likely to be both specific to the tumour and present in substantially every tumour cell.

In one aspect the neoantigen may be a clonal neoantigen.

A “clonal neoantigen” (also sometimes referred to as a “truncal neoantigen”) is a neoantigen arising from a clonal mutation. A “clonal mutation” (sometimes referred to as a “truncal mutation”) is a mutation that is present in essentially every tumour cell in one or more samples from a subject (or that can be assumed to be present in essentially every tumour cell from which the tumour genetic material in the sample(s) is derived). Thus, a clonal mutation may be a mutation that is present in every tumour cell in one or more samples from a subject. For example, a clonal mutation may be a mutation which occurs early in tumorigenesis.

A “subclonal neoantigen” (also sometimes referred to as a “branched neoantigen”) is a neoantigen arising from a subclonal mutation. A “subclonal mutation” (also sometimes referred to as a “branch mutation”) is a mutation that is present in a subset or a proportion of cells in one or more tumour samples from a subject (or that can be assumed to be present in a subset of the tumour cells from which the tumour genetic material in the sample(s) is derived). For example, a subclonal mutation may be the result of a mutation occurring in a particular tumour cell later in tumorigenesis, which is found only in cells descended from that cell.

The wording “essentially every tumour cell” in relation to one or more samples of a subject may refer to at least 70%, at least 75%, at least 80%, at least 85%, at least 90%, at least 91%, at least 92%, at least 93%, at least 94% at least 95%, at least 96%, at least 97%, at least 98%, or at least 99% of the tumour cells in the one or more samples or the subject.

As such, a clonal neoantigen is a neoantigen which is expressed effectively throughout a tumour. A subclonal neoantigen is a neoantigen that is expressed in a subset or a proportion of cells or regions in a tumour. ‘Expressed effectively throughout a tumour’ may mean that the clonal neoantigen is expressed in all regions of the tumour from which samples are analysed.

It will be appreciated that a determination that a mutation is ‘encoded (or expressed) within essentially every tumour cell’ refers to a statistical calculation and is therefore subject to statistical analysis and thresholds.

Likewise, a determination that a clonal neoantigen is ‘expressed effectively throughout a tumour’ refers to a statistical calculation and is therefore subject to statistical analysis and thresholds.

Various methods for determining whether a neoantigen is “clonal” or “subclonal” are known in the art. Any suitable method may be used to identify a clonal neoantigen.

By way of example, the cancer cell fraction (CCF), describing the proportion of cancer cells that harbour a mutation, may be used to determine whether mutations are clonal or subclonal. For example, the cancer cell fraction may be determined by integrating variant allele frequencies with copy numbers and purity estimates as described by Landau et al. (Cell. 2013 Feb. 14; 152(4):714-26).

Suitably, CCF values may be calculated for all mutations identified within each and every tumour region analysed. If only one region is used (i.e. only a single sample), only one set of CCF values will be obtained. This will provide information as to which mutations are present in all tumour cells within that tumour region and will thereby provide an indication if the mutation is clonal or subclonal.

Such a CCF estimate can also be used to identify mutations that are likely to be clonal. A clonal mutation may be defined as a mutation which has a cancer cell fraction (CCF)≥0.75, such as a CCF≥0.80, 0.85. 0.90, 0.95 or 1.0. A subclonal mutation may be defined as a mutation which has a CCF<0.95, 0.90, 0.85, 0.80, or 0.75. In one aspect, a clonal mutation is defined as a mutation which has a CCF≥0.95 and a subclonal mutation is defined as a mutation which has a CCF<0.95.

As stated, identifying a clonal mutation is subject to statistical analysis and threshold. A CCF estimate may be associated with (e.g. derived from) a distribution associating a probability with each of a plurality of possible values of CCF between 0 and 1, from which statistical estimates of confidence may be obtained. For example, a mutation may be defined as likely to be a clonal mutation if the 95% CCF confidence interval is >=0.75, i.e. the upper bound of the 95% confidence interval of the estimated CCF is greater than or equal to 0.75. In other words, a mutation may be defined as likely to be a clonal mutation if there is an interval of CCF with lower bound L and upper bound H that is such that P(L<CCF<H)=95% with H>=0.75. Alternatively, a mutation may be identified as clonal if P(CCF>0.75)>=0.5.

In one aspect a mutation may be defined as a clonal mutation if the 95% confidence interval of the CCF includes CCF=1.

In another aspect a mutation may be identified as clonal if there is more than a 50% chance or probability that its cancer cell fraction (CCF) reaches or exceeds the required value as defined above, for example 0.75 or 0.95, such as a chance or probability of 55%, 60%, 65%, 70%, 75%, 80%, 85%, 90%, 95% or more.

Probability values may be expressed as percentages or fractions. The probability may be defined as a posterior probability.

In one aspect, a mutation may be identified as clonal if the probability that the mutation has a cancer cell fraction greater than 0.95 is 0.75.

In another aspect, a mutation may be identified as clonal if there is more than a 50% chance that its cancer cell fraction (CCF) is 0.95.

In a further aspect, mutations may be classified as clonal or subclonal based on whether the posterior probability that their CCF exceeds a first threshold (e.g., 0.95) is greater or lesser than a second threshold (e.g., 0.5), respectively.

In another aspect a mutation may be identified as clonal if the probability that the mutation has a cancer cell fraction greater than 0.75 is 0.5.

In one aspect the T cell therapy may comprise T cells which target a plurality i.e. more than one clonal neo-antigen.

In one aspect the number of clonal neoantigens is 2-1000. For example, the number of clonal neoantigens may be 2, 3, 4, 5, 6, 7, 8, 9, 10, 20, 50, 100, 150, 200, 250, 300, 350, 400, 450, 500, 550, 600, 650, 700, 750, 800, 850, 900, 950 or 1000, for example the number of clonal neoantigens may be from 2 to 100.

In one aspect, the T cell therapy as described herein may comprise a plurality or population, i.e., more than one, of T cells wherein the plurality of T cells comprises a T cell which recognises a clonal neoantigen and a T cell which recognises a different clonal neoantigen. As such, the T cell therapy comprises a plurality of T cells which recognise different clonal neoantigens.

In one aspect the number of clonal neoantigens recognised by the plurality of T cells is 2-1000. For example, the number of clonal neoantigens recognised may be 2, 3, 4, 5, 6, 7, 8, 9, 10, 20, 50, 100, 150, 200, 250, 300, 350, 400, 450, 500, 550, 600, 650, 700, 750, 800, 850, 900, 950 or 1000, for example the number of clonal neoantigens recognised may be from 2 to 100.

In one aspect the plurality of T cells recognises the same clonal neoantigen.

In one aspect the neoantigen may be a subclonal neoantigen as described herein.

As described above, a clonal neoantigen is one which is encoded within essentially every tumour cell, that is the mutation encoding the neoantigen is present within essentially every tumour cell and/or is expressed effectively throughout the tumour. However, a clonal neoantigen may be predicted to be presented by an HLA molecule encoded by an HLA allele which is lost in at least part of a tumour. In this case, the clonal neoantigen may not actually be presented on essentially every tumour cell. As such, the presentation of the neoantigen may not be clonal, i.e. it is not presented within essentially every tumour cell. Methods for predicting loss of HLA are described in International Patent Publication No. WO2019/012296.

In one aspect of the invention as described herein the neoantigen is predicted to be presented within essentially every tumour cell (i.e. the presentation of the neoantigen is clonal).

### Neoantigen-Specific T Cell Therapy

The T cell therapy according to the invention may comprise T cells which target neoantigens. In one aspect of the invention, the T cell therapy may comprise T cells which target clonal neoantigens. In the context of the present invention, the term “target” may mean that the T cell is specific for, and mounts a response to, the neoantigen.

In one aspect the T cell therapy may comprise T cells which have been selectively expanded to target neoantigens, such as clonal neoantigens.

That is, the T cell therapy may have an increased number of T cells that target one or more neoantigens. For example, the T cell population of the invention may have an increased number of T cells that target a neoantigen compared with the T cells in the sample isolated from the subject. That is to say, the composition of the T cell population will differ from that of a “native” T cell population (i.e. a population that has not undergone the identification and expansion steps discussed herein), in that the percentage or proportion of T cells that target a neoantigen will be increased, and the ratio of T cells in the population that target neoantigens to T cells that do not target neoantigens will be higher in favour of the T cells that target neoantigens.

The T cell population according to the invention may have at least about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100% T cells that target a neoantigen (which may be a respective neoantigen). For example, the T cell population may have about 0.2%-5%, 5%-10%, 10-20%, 20-30%, 30-40%, 40-50%, 50-70% or 70-100% T cells that target a neoantigen. In one aspect the T cell population has at least about 1, 2, 3, 4 or 5% T cells that target a neoantigen, for example at least about 2% or at least 2% T cells that target a neoantigen.

Alternatively put, the T cell population may have not more than about 5, 10, 15, 20, 25, 30, 35, 40, 45, 50, 55, 60, 65, 70, 75, 80, 85, 90, 91, 92, 93, 94, 95, 96, 97, 98, 99, 99.1, 99.2, 99.3, 99.4, 99.5, 99.6, 99.7, 99.8% T cells that do not target a neoantigen. For example, the T cell population may have not more than about 95%-99.8%, 90%-95%, 80-90%, 70-80%, 60-70%, 50-60%, 30-50% or 0-30% T cells that do not target a neoantigen. In one aspect the T cell population has not more than about 99, 98, 97, 96 or 95% T cells that do not target a neoantigen, for example not more than about 98% or 95% T cells that do not target a neoantigen.

An expanded population of neoantigen-reactive T cells may have a higher activity than a population of T cells not expanded, for example, using a neoantigen peptide. Reference to “activity” may represent the response of the T cell population to restimulation with a neoantigen peptide, e.g. a peptide corresponding to the peptide used for expansion, or a mix of neoantigen peptides. Suitable methods for assaying the response are known in the art. For example, cytokine production may be measured (e.g. TNFα, IL2 or IFNγ production may be measured). The reference to a “higher activity” includes, for example, a 1-5, 5-10, 10-20, 20-50, 50-100, 100-500, 500-1000-fold increase in activity. In one aspect the activity may be more than 1000-fold higher.

The T cell population may be all or primarily composed of CD8+ T cells, or all or primarily composed of a mixture of CD8+ T cells and CD4+ T cells or all or primarily composed of CD4+ T cells.

In particular aspects, the T cells in the T cell therapy may be generated from T cells isolated from one or more samples of a subject with a tumour. The sample may be a tumour sample, a peripheral blood sample (e.g. PBMCs) or a sample from other tissues of the subject.

The T cells may be generated from a sample from the tumour in which the neo-antigen is identified. In other words, the T cell population may be isolated from a sample derived from the tumour of a patient to be treated. Such T cells are referred to herein as ‘tumour infiltrating lymphocytes’ (TILs).

T cells may be isolated using methods which are well known in the art. For example, T cells may be purified from single cell suspensions generated from samples on the basis of expression of CD3, CD4 or CD8 or other relevant markers. T cells may be enriched from samples by passage through a Ficoll-paque gradient.

As described herein, in one aspect of the invention the T cells are expanded prior to batch release.

Expansion of T cells may be performed using methods which are known in the art. For example, T cells may be expanded by ex vivo culture in conditions which are known to provide mitogenic stimuli for T cells. By way of example, the T cells may be cultured with cytokines such as IL-2 or with mitogenic antibodies such as anti-CD3 and/or CD28. The T cells may also be co-cultured with feeder cells, such as peripheral blood mononuclear cells (PBMC) or antigen-presenting cells (APCs). In one aspect, the APCs are irradiated. In another aspect, the APCs are dendritic cells. The dendritic cells may be derived from monocytes obtained from the patient's blood, referred to herein as monocyte-derived dendritic cells (MoDCs).

In one aspect the T cells are expanded in the presence of an antigen. The antigen may be a peptide. The peptide may be displayed by an APC, such as a dendritic cell.

Thus, in one aspect of the invention, T cells that are capable of specifically recognising one or more neoantigens are identified in a sample from the subject and then expanded by ex vivo culture as described herein. Identification of neoantigen-specific T cells in a mixed starting population of T cells may be performed using methods which are known in the art. In embodiments, T cell reactivity to the specific antigen (such as a neoantigen) may be assessed, e.g. as shown in the present Examples.

Alternatively, neoantigen-specific T cells may be identified using MHC multimers comprising a neoantigen peptide. Antigens may also be presented by a soluble MHC multimer as described herein. Antigens may also be presented by MHC on the surface of another cell type such as a dendritic cell or B cell or autologous cell line.

MHC multimers are oligomeric forms of MHC molecules, designed to identify and isolate T-cells with high affinity to specific antigens amid a large group of unrelated T-cells. Multimers may be used to display class 1 MHC, class 2 MHC, or nonclassical molecules (e.g. CD1d).

The most commonly used MHC multimers are tetramers. These are typically produced by biotinylating soluble MHC monomers, which are typically produced recombinantly in eukaryotic or bacterial cells. These monomers then bind to a backbone, such as streptavidin or avidin, creating a tetravalent structure. These backbones are conjugated with fluorochromes to subsequently isolate bound T-cells via flow cytometry, for example.

In another aspect of the invention, the T cells undergo a specific expansion step, whereby T cells that respond to the one or more neoantigens are expanded in preference to other T cells in the starting material that do not respond to the neoantigen(s). This may be achieved by co-culturing the T cells with antigen-presenting cells (APCs) which present the relevant neoantigen(s). The APCs may be pulsed with peptides containing the identified mutations as single stimulants or as pools of stimulating neoantigens or peptides. Alternatively, the APCs may be modified to express the neoantigen sequence(s), for example by transfecting the APCs with DNA/RNA molecule(s) (such as e.g. mRNA molecule(s)) encoding the neoantigen sequence(s).

Other suitable methods for said expansion will be known to those of skill in the art. For example, International Patent Publication No. WO2019/094642 describes a number of protocols for expansion of T cells in response to neoantigens.

### T Cell Expansion

In one aspect of the invention, T cells may be expanded by methods that use reduced concentrations of IL-2. The concentration of IL-2 used in the antigen-specific expansion of T cells may be described as “lower” or “reduced”, for example in comparison to the concentration of IL-2 used in a typical rapid expansion step. The lower concentration of IL-2 is used to promote selective expansion of the T cells in response to antigen and reduce non-specific expansion.

By way of example, typical TIL expansion protocols use very high, non-physiological levels of IL-2 in the rapid expansion step. For example, WO 2018/182817 discloses a method of expanding TIL that uses an IL-2 concentration of about 1,000 to about 10,000 IU/ml, for example 3,000 IU/ml of IL-2, in the rapid expansion step.

In contrast, according to the present invention, the T cell therapy may be, or may have been, produced by an expansion method that uses IL-2 at a concentration in the range of from about 10 IU/ml to about 1,000 IU/ml, for example from about 25 IU/ml to about 500 IU/ml, such as from about 50 IU/ml to about 250 IU/ml, preferably from about 75 IU/ml to about 125 IU/ml. The concentration of IL-2 used in a T cell expansion step may therefore be about 10, 25, 50, 75, 100, 125, 150, 200, 250, 300, 400, 500, 600, 700, 800, 900 or 1,000 IU/ml. In one aspect the method may use IL-2 at a concentration of less than about 1,000 IU/ml.

In one aspect the T cells may be pre-expanded, for example prior to co-culture with APCs.

In one embodiment, pre-expanded T cells, for example TIL, may be combined with APCs and co-cultured with IL-2 at a concentration of from 50 IU/ml to 500 IU/ml, such as 75 IU/ml to 150 IU/ml, preferably about 100 IU/ml, in order to produce the therapeutic T cell product. The IL-2 concentration may remain constant throughout the culture step, for example by controlling the concentration with repeated feeding steps, or may vary throughout the culture without exceeding the maximum concentration specified. In one aspect the APCs are dendritic cells, for example, mature dendritic cells (mDC).

It is hypothesized that T cell products that have been expanded in vitro using reduced concentrations of IL-2 as defined above will advantageously require lower doses of IL-2 in vivo in order to persist and engraft.

In one aspect of the invention as described herein said T cell therapy may comprise T cells that have been expanded in the presence of IL-2 at a concentration of less than about 1,000 IU/ml, preferably in the presence of IL-2 at a concentration of about 100 IU/ml.

### Cancer

In one aspect the cancer as described herein is a neoplasm arising from cells of the central or peripheral nervous system, cardiovascular systemic (including the lymphatic system), gastrointestinal tract, respiratory system, genitourinary system, endocrine system (including the neuroendocrine system), exocrine system, reproductive system, haemotological and immune systems, musculoskeletal system, and cancers of unknown primary (CUP). This includes but is not limited to cancers referred to as lung cancer (small cell, non-small cell and mesothelioma), bronchial cancer, skin cancer (e.g. melanoma), bladder cancer, gastric cancer, oesophageal cancer, salivary gland cancer, breast cancer (e.g. triple negative breast cancer), thymus cancer, colorectal cancer, vaginal cancer, cervical cancer, ovarian cancer, endometrial cancer, penile cancer, testicular cancer, kidney cancer (renal cell), brain cancer (eg. gliomas, astrocytomas, glioblastomas), lymphoma, small bowel/intestinal cancers (duodenal and jejunal), haematological malignancies (e.g. leukaemia, multiple myeloma), liver/hepatic cancer (hepatocellular carcinoma), pancreatic cancer, hepatobiliary tumours, germ cell cancers, bone marrow cancer, bone cancer, prostate cancer, merkel cell carcinoma, head and neck cancers (squamous cell), thyroid cancer, high microsatellite instability (MSI-H), and sarcomas (including cancers with sarcomatoid components, e.g. leiomyosarcoma, myosarcoma).

In one aspect the cancer is selected from melanoma and non-small cell lung cancer (NSCLC).

In one aspect the cancer, such as melanoma or NSCLC, may be metastatic, and/or inoperable and/or recurrent.

Treatment according to the present invention may also encompass targeting circulating tumour cells and/or metastases derived from the tumour.

### Subject

The terms “subject” and “patient” are used interchangeably herein.

In a preferred aspect of the present invention, the subject is a mammal, preferably a cat, dog, horse, donkey, sheep, pig, goat, cow, mouse, rat, rabbit or guinea pig, but most preferably the subject is a human.

As defined herein “treatment” refers to reducing, alleviating or eliminating one or more symptoms of the disease which is being treated, relative to the symptoms prior to treatment.

“Prevention” (or prophylaxis) refers to delaying or preventing the onset of the symptoms of the disease. Prevention may be absolute (such that no disease occurs) or may be effective only in some individuals or for a limited amount of time.

### Combination Therapies

The T cell therapy as described herein may also be combined with other suitable therapies.

The methods and uses for treating cancer according to the present invention may be performed in combination with additional cancer therapies. In particular, the T cell compositions according to the present invention may be administered in combination with immune checkpoint intervention, co-stimulatory antibodies, chemotherapy and/or radiotherapy, targeted therapy or monoclonal antibody therapy.

Immune checkpoint molecules include both inhibitory and activatory molecules, and interventions may apply to either or both types of molecule. Immune checkpoint inhibitors include, but are not limited to, PD-1 inhibitors, PD-L1 inhibitors, Lag-3 inhibitors, Tim-3 inhibitors, TIGIT inhibitors, BTLA inhibitors and CTLA-4 inhibitors, for example. Co-stimulatory antibodies deliver positive signals through immune-regulatory receptors including but not limited to ICOS, CD137, CD27 OX-40 and GITR.

Examples of suitable immune checkpoint interventions which prevent, reduce or minimize the inhibition of immune cell activity include pembrolizumab, nivolumab, atezolizumab, durvalumab, avelumab, tremelimumab and ipilimumab.

A chemotherapeutic entity as used herein refers to an entity which is destructive to a cell, that is the entity reduces the viability of the cell. The chemotherapeutic entity may be a cytotoxic drug. A chemotherapeutic agent contemplated includes, without limitation, alkylating agents, anthracyclines, epothilones, nitrosoureas, ethylenimines/methylmelamine, alkyl sulfonates, alkylating agents, antimetabolites, pyrimidine analogs, epipodophylotoxins, enzymes such as L-asparaginase; biological response modifiers such as IFNα, IL-2, G-CSF and GM-CSF; platinum coordination complexes such as cisplatin, oxaliplatin and carboplatin, anthracenediones, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

‘In combination’ may refer to administration of the additional therapy before, at the same time as or after administration of the T cell composition according to the present invention.

In addition or as an alternative to the combination with checkpoint blockade, the T cell composition of the present invention may also be genetically modified to render them resistant to immune-checkpoints using gene-editing technologies including but not limited to TALEN and Crispr/Cas. Such methods are known in the art, see e.g. US20140120622. Gene editing technologies may be used to prevent the expression of immune checkpoints expressed by T cells including but not limited to PD-1, Lag-3, Tim-3, TIGIT, BTLA CTLA-4 and combinations of these. The T cell as discussed here may be modified by any of these methods.

The T cell according to the present invention may also be genetically modified to express molecules increasing homing into tumours and or to deliver inflammatory mediators into the tumour microenvironment, including but not limited to cytokines, soluble immune-regulatory receptors and/or ligands.

### Composition

The T cell therapy as described herein may be provided in the form of a composition.

The composition may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Compositions, according to the current invention, are administered using any amount and by any route of administration effective for preventing or treating a subject. An effective amount refers to a sufficient amount of the composition to beneficially prevent or ameliorate the symptoms of the disease or condition.

The exact dosage is chosen by the individual physician in view of the patient to be treated. Dosage and administration are adjusted to provide sufficient levels of the active agent(s) or to maintain the desired effect in a subject. Additional factors which may be taken into account include the severity of the disease state, e.g., liver function, cancer progression, and/or intermediate or advanced stage of macular degeneration; age; weight; gender; diet, time; frequency of administration; route of administration; drug combinations; reaction sensitivities; level of immunosuppression; and tolerance/response to therapy. Long acting pharmaceutical compositions are administered, for example, hourly, twice hourly, every three to four hours, daily, twice daily, every three to four days, every week, or once every two weeks depending on half-life and clearance rate of the particular composition.

The active agents of the pharmaceutical compositions of embodiments of the invention are preferably formulated in dosage unit form for ease of administration and uniformity of dosage. The expression “dosage unit form” as used herein refers to a physically discrete unit of active agent appropriate for the patient to be treated. The total daily usage of the compositions of the present invention will be decided by the attending physician within the scope of sound medical judgment. For any active agent, the therapeutically effective dose is estimated initially either in cell culture assays or in animal models, potentially mice, pigs, goats, rabbits, sheep, primates, monkeys, dogs, camels, or high value animals. The cell-based, animal, and in vivo models provided herein are also used to achieve a desirable concentration, total dosing range, and route of administration. Such information is used to determine useful doses and routes for administration in humans.

A therapeutically effective dose refers to that amount of active agent that ameliorates the symptoms or condition or prevents progression of the disease or condition. Therapeutic efficacy and toxicity of active agents are determined by standard pharmaceutical procedures in cell cultures or experimental animals, e.g., ED₅₀ (dose therapeutically effective in 50% of the population) and LD₅₀ (dose lethal to 50% of the population). The dose ratio of toxic to therapeutic effects is the therapeutic index, which is expressed as the ratio, LD₅₀/ED₅₀. Pharmaceutical compositions having large therapeutic indices are preferred. The data obtained from cell culture assays and animal studies are used in formulating a range of dosage for human use.

As formulated with an appropriate pharmaceutically acceptable carrier in a desired dosage, the pharmaceutical composition or methods provided herein is administered to humans and other mammals for example topically for skin tumours (such as by powders, ointments, creams, or drops), orally, rectally, mucosally, sublingually, parenterally, intracisternally, intravaginally, intraperitoneally, intravenously, subcutaneously, bucally, sublingually, ocularly, or intranasally, depending on preventive or therapeutic objectives and the severity and nature of the cancer-related disorder or condition.

Injections of the pharmaceutical composition include intravenous, subcutaneous, intra-muscular, intraperitoneal, or intra-ocular injection into the inflamed or diseased area directly, for example, for esophageal, breast, brain, head and neck, and prostate inflammation. In one aspect, the pharmaceutical composition described herein is administered intravenously.

Liquid dosage forms are, for example, but not limited to, intravenous, ocular, mucosal, pharmaceutically acceptable emulsions, microemulsions, solutions, suspensions, syrups, and elixirs. In addition to at least one active agent, the liquid dosage forms potentially contain inert diluents commonly used in the art such as, for example, water or other solvents; solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols, fatty acid esters of sorbitan, and mixtures thereof. Besides inert diluents, the ocular, oral, or other systemically-delivered compositions also include adjuvants such as wetting agents, emulsifying agents, and suspending agents.

Dosage forms for topical or transdermal administration of the pharmaceutical composition herein include ointments, pastes, creams, lotions, gels, powders, solutions, sprays, inhalants, or patches. The active agent is admixed under sterile conditions with a pharmaceutically acceptable carrier. Preservatives or buffers may be required. For example, ocular or cutaneous routes of administration are achieved with aqueous drops, a mist, an emulsion, or a cream. Administration is in a therapeutic or prophylactic form. Certain embodiments of the invention herein contain implantation devices, surgical devices, or products which contain disclosed compositions (e.g., gauze bandages or strips), and methods of making or using such devices or products. These devices may be coated with, impregnated with, bonded to or otherwise treated with the composition herein.

Transdermal patches have the added advantage of providing controlled delivery of the active ingredients to the eye and body. Such dosage forms can be made by dissolving or dispensing the compound in the proper medium. Absorption enhancers are used to increase the flux of the compound across the skin. Rate is controlled by either providing a rate controlling membrane or by dispersing the compound in a polymer matrix or gel.

Injectable preparations of the pharmaceutical composition, for example, sterile injectable aqueous or oleaginous suspensions are formulated according to the known art using suitable dispersing agents, wetting agents, and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or a suspending medium. For this purpose, bland fixed oil including synthetic mono-glycerides or di-glycerides is used. In addition, fatty acids such as oleic acid are used in the preparation of injectables. The injectable formulations are sterilized prior to use, for example, by filtration through a bacterial-retaining filter, by irradiation, or by incorporating sterilizing agents in the form of sterile solid compositions, which are dissolved or dispersed in sterile water or other sterile injectable medium. Slowing absorption of the agent from subcutaneous or intratumoral injection was observed to prolong the effect of an active agent. Delayed absorption of a parenterally administered active agent is accomplished by dissolving or suspending the agent in an oil vehicle. Injectable depot forms are made by forming microencapsulated matrices of the agent in biodegradable polymers such as polylactide-polyglycolide. Depending upon the ratio of active agent to polymer and the nature of the particular polymer employed, the rate of active agent release is controlled. Examples of other biodegradable polymers include poly (orthoesters) and poly(anhydrides). Depot injectable formulations are also prepared by entrapping the agent in liposomes or microemulsions that are compatible with body tissues.

Solid dosage forms for oral administration include capsules, tablets, pills, powders, and granules. In solid dosage forms, the active agent is mixed with at least one inert, pharmaceutically acceptable excipient or carrier such as sodium citrate, dicalcium phosphate, fillers, and/or extenders such as starches, sucrose, glucose, mannitol, and silicic acid; binders such as carboxymethylcellulose, alginates, gelatin, polyvinylpyrrolidinone, sucrose, and acacia; humectants such as glycerol; disintegrating agents such as agar-agar, calcium carbonate, potato or tapioca starch, alginic acid, certain silicates, and sodium carbonate; solution retarding agents such as paraffin; absorption accelerators such as quaternary ammonium compounds; wetting agents, for example, cetyl alcohol and glycerol monostearate; absorbents such as kaolin and bentonite clay; and lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using excipients such as milk sugar as well as high molecular weight PEG and the like. The solid dosage forms of tablets, dragees, capsules, pills, and granules are prepared with coatings and shells such as enteric coatings, release controlling coatings, and other coatings known in the art of pharmaceutical formulating. In these solid dosage forms, the active agent(s) are admixed with at least one inert diluent such as sucrose or starch. Such dosage forms also include, as is standard practice, additional substances other than inert diluents, e.g., tableting lubricants and other tableting aids such as magnesium stearate and microcrystalline cellulose. In the case of capsules, tablets and pills, the dosage forms may also include buffering agents. The composition optionally contains opacifying agents that release the active agent(s) only, preferably in a certain part of the intestinal tract, and optionally in a delayed manner. Examples of embedding compositions include polymeric substances and waxes.

Also described herein is a method of treating a subject that has been diagnosed as having cancer, the method comprising the following steps: a) providing a sample of a pharmaceutical product comprising T cells, b) analysing the reactivity of the sample T cells to an assay antigen; c) determining that said sample T cells meet a predetermined threshold for reactivity to the assay antigen; and d) if the sample T cells meet the predetermined threshold, administering the pharmaceutical product to the subject, wherein the pharmaceutical product comprises tumour infiltrating lymphocytes (TILs) isolated from a tumour sample from the subject.

In some embodiments, the assay antigen described herein is a tumour associated antigen (TAA) or tumour specific antigen (TSA). In some embodiments, the assay antigen described herein is a neoantigen. In some embodiments, the assay antigen described herein is a clonal neoantigen.

In some embodiments, step b) of the method described herein is carried out in the absence of antigen presenting cells (APCs). For example, the T cells may present said assay antigen to other T cells (T-to-T cell or T:T cell assay). Moreover, step b) of the method described herein can be carried out in the absence of any other cells.

In some embodiments, the reactivity of the T cells to the assay antigen is analysed by measuring expression level of at least one cytokine and/or measuring expression level of at least one T cell marker or T cell surface marker. For example, at least one cytokine may comprise IFN-γ and/or TNF-α.

Additionally and/or alternatively, at least one T cell marker or T cell surface marker may comprise 4-1BB, CD25, OX40, Ki67, Granzyme B, Perforin, CD107a, LAG-3, PD-1, TIM-3, CTLA4, CD39, Fas, FasL, CD40L, KLRG1, GITR and ICOS.

The expression level of at least one cytokine and/or at least one T cell marker or T cell surface marker can be determined by flow cytometry, immunoassay, immunoassay, ELISpot and/or TCR sequencing. Reactive T cell count versus non-reactive T cells can be calculated by flow cytometry by subtracting the negative control (e.g. DMSO) results from that of the peptides result.

In some embodiments, the assay antigen of the method described herein is identified prior to or after T cell expansion.

In some embodiments, the pharmaceutical product of the method described herein comprises T cells that have been expanded in vitro.

The T cells of the pharmaceutical product may have been expanded in the presence of an expansion antigen prior to providing the sample for the analysing of reactivity.

In some embodiments, the assay antigen and the expansion antigen are the same antigen.

In other embodiments, the assay antigen and the expansion antigen are different antigens.

In some embodiments, the threshold of reactivity is met when the number of reactive T cells is at least 1×10⁵ reactive cells.

Preferably the threshold of reactivity is met when the number of reactive T cells are between from 1×10⁷ to 1×10⁹ reactive cells

In some embodiments, the pharmaceutical product of the method described herein comprises engineered T cells. For example, engineered TILs.

In some embodiments, the method described herein is for the treatment of cancer by immunotherapy where the cancer is melanoma or non-small cell lung cancer (NSCLC).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of aspects of this disclosure. Numeric ranges are inclusive of the numbers defining the range.

The headings provided herein are not limitations of the various aspects or aspects of this disclosure which can be had by reference to the specification as a whole. Accordingly, the terms defined immediately below are more fully defined by reference to the specification as a whole.

The term “protein”, as used herein, includes proteins, polypeptides, and peptides.

Other definitions of terms may appear throughout the specification. Before the exemplary aspects are described in more detail, it is to understand that this disclosure is not limited to particular aspects described, as such may, of course, vary. It is also to be understood that the terminology used herein is for the purpose of describing particular aspects only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be described, by way of example only, with reference to the following Examples.

### EXAMPLES

### Example 1

Two open-label, multi-centre, phase I/IIa studies are being carried out to characterise the safety and clinical activity of autologous, expanded clonal neo-antigen-reactive T cells (cNeT) administered intravenously in adult patients with either advanced inoperable or metastatic non-small cell lung cancer (NSCLC) (NCT04032847) or metastatic or recurrent melanoma (NCT03997474).

### Tissue Procurement

Tumour and blood samples procured from the patient are shipped to the manufacturing site for further processing. The tumour and blood samples are sequenced and analysed to identify clonal neoantigens. Using this information, clonal neoantigen peptides are subsequently manufactured. Tumour infiltrating lymphocytes (TIL) are isolated from the tumour tissue. The blood sample is used to manufacture dendritic cells which can process and present the clonal neoantigen peptides to the TIL. The isolated and pre-expanded TIL are combined with the dendritic cells which have been pulsed with the clonal neoantigen peptides. In this way, clonal neoantigen T cells (cNeT) are specifically isolated and expanded. The cNeT cells are harvested and formulated to form ATL001.

### Study Treatment

All patients will receive a non-myeloablative lymphodepletion regimen of fludarabine 30 mg/m² i.v. followed by cyclophosphamide 300 mg/m² i.v. on each of Days −6, −5, and −4 prior to cell infusion.

Eligible patients will receive a single intravenous infusion of ATL001. The cell dose to be administered will be 1×10⁷ CD3+ cells. The maximum dose in a 30 ml infusion bag is 1×10⁹ CD3+ cells.

Patients will receive 10 doses of IL-21 MIU/m² s.c. daily from days 0-9 of the study, starting approximately 3 hours post-infusion.

Patients will stay in hospital over this treatment period.

### Post Treatment Assessments

Following discharge from hospital patients will attend study visits on study days 14, 21 and 28 then at week 6, 12, 18 and 24, and then every 12 weeks until week 104. Safety will be assessed by regular assessments of infusion reactions, adverse events, physical examinations, ECOG status, laboratory tests, vital signs, electrocardiograms, and concomitant medication usage. The severity of AEs will be assessed using National Cancer Institute Common Terminology Criteria for Adverse Events (Version 5.0). Clinical activity will be assessed by CT scans every 6 weeks to week 24 and then every 12 weeks.

### Study Design

Following consent and screening, eligible patients will initially enter the study for procurement of tumour tissue and blood to manufacture ATL001. Tumour tissue may be procured either before or after receiving standard systemic therapies. While ATL001 is being manufactured, patients will receive standard therapy.

### Study Objectives and Outcome Measures

The primary objective of the study is to describe the safety and tolerability of the study product, assessed by the frequency and severity of adverse events (AEs) and serious adverse events (SAEs) following tissue procurement and administration of lymphodepletion agents, ATL001 and IL-2.

The secondary clinical efficacy endpoints include percentage change from baseline in tumour size, objective response rate (ORR), time to response (TTR), duration of response (DoR), disease control rate (CR+PR+ durable SD), progression free survival (PFS) and overall survival (OS). RECIST v1.1 and imRECIST criteria will be applied.

The exploratory objectives of the study include evaluation of the persistence, phenotype and functionality of cNeT cells and possible relationships with clinical outcomes, the evaluation of potential biomarkers of clinical activity and factors affecting response, and the evaluation of factors that may affect the quality of ATL001.

Blood samples are taken from patients at multiple time points before and after ATL001 administration, at days −6 (pre lymphodepletion), 0 (pre administration), 3, 7, 10, 14, 21 and 28 then at 6 weeks, 12 weeks, 18 weeks and 24 weeks then every 3 months until progression. These blood samples will be utilised for a number of different assays including TCR sequencing to track the TCR that were present in the ATL001 product to see if expansion of specific clones can be observed in the blood of the patient. In addition, samples will be taken to allow for detection and analysis of circulating tumour DNA.

Further blood samples will be taken into heparin at each time point. These will be utilised in a whole blood flow cytometry assay to enumerate key immune cells within the blood. PBMC will then be isolated. The PBMC will be then used in several assays: ELISPOT to determine if reactivity to the neoantigen peptides can be detected ex vivo, and intracellular cytokine staining to determine the phenotype of the responding cells. An extended phenotyping panel using flow cytometry will also be used in order to determine the memory phenotype of the T cells (by looking at CD27, CD28 CD45RA and CCR7 expression), any exhaustion markers that may be present (such as CD57, PD-1, TIM3) and a panel that looks at CD25 and FoxP3 expression to determine the number of T regs present.

### Results

We have analysed data from the first six patients in the two ongoing clinical trials, three patients with NSCLC and three with melanoma. Patients had received a median of 2.5 lines of therapy prior to receiving cNeT. All had progressive disease at the time of lymphodepletion prior to cNeT infusion and each patient completed their first scheduled scan six weeks post-cNeT infusion to assess tumor size. Data from these six patients has demonstrated a favourable cNeT tolerability profile and provided encouraging initial evidence of cNeT engraftment.

cNeT Tolerability

Overall, the tolerability profile of cNeT was observed to be similar to that of standard TIL products that have not been enriched for cNeT reactivities, with the lymphodepletion regimen accounting for most of the observed higher-grade adverse events, being neutropenia, and febrile neutropenia/neutropenic sepsis. We observed no grade 3 or 4 toxicities reported as causally related to IL-2. We observed two serious adverse events, or SAEs, that were deemed related or possibly related to ATL001. The first was an instance of immune effector cell-associated neurotoxicity syndrome. The event was also deemed potentially related to IL-2. The patient was treated with dexamethasone and tocilizumab and their acute condition improved. The patient, however, subsequently died due to progression of the underlying cancer. The second SAE was a non-specific encephalopathy (grade 1), which led to hospitalization. The episode of encephalopathy responded to corticosteroids and the patient was discharged from the hospital and continued on the trial. Two additional patients subsequently died due to progression of the underlying cancer.

A formal review of safety was conducted by an Independent Data and Safety Monitoring Committee to review the data from these first six patients. The Data and Safety Monitoring Committee recommended that the two clinical trials should continue as planned with no required modifications.

cNeT Activity

We observed stable disease at six-weeks post-dosing in four out of the six patients and progressive disease in two patients. One patient had a reduction in the size of two of their four tumor lesions by approximately 55% and 90%. Engraftment data for our cNeT are currently available from six patients, with evidence of engraftment being observed in three patients, and the highest engraftment observed in the patient who received the highest cNeT dose. It has been observed in prior studies of CAR-T cell therapies that engraftment and expansion of tumor-reactive T cells post infusion is correlated to clinical response. This correlation has not been evaluable with prior TIL therapies due to the lack of routine characterization of the active component of the infused cells, and the associated inability to track the active component post-dosing. Since we characterize our cell product candidates at the level of individual cNeT reactivities, we are able to determine engraftment, peak expansion, and durability of persistence of clonal neoantigen-reactive T cells. An additional benefit of our detailed product characterization is the ability to demonstrate the polyclonality of both the infused product and the engrafted cells. We have identified between two and 28 unique clonal neoantigen reactivities in individual patient cNeT product candidates in both our clinical trials and have demonstrated the presence of the same polyclonal cNeT reactivities following infusion in both patients in whom engraftment was observed.

### Example 2

Patient T-05 enrolled in the melanoma trial with an initial diagnosis of BRAF wild type cutaneous melanoma in 2006. The patient had previously received a three-cycle combination of ipilimumab in 2017, which was discontinued due to toxicity. The patient remained off treatment and had recurrent cutaneous lesions resected in the years following immunotherapy. A soft tissue lesion was excised from the patient's abdomen in February 2020 and was taken forward into cNeT manufacturing.

### Intracellular Cytokine Secretion Assay

Intracellular cytokine staining (ICS) is used to assess cNeT cell function (potency) by measuring the ability of the cell population to produce the effector cytokines IFN-γ and/or TNF-α after stimulation with peptides corresponding to patient specific neoantigens.

The ICS assay requires 0.1×10⁶ cNeT for seeding and stimulation for 16-18 hour at 37° C., in the presence of the protein transport inhibitors Brefeldin A and Monensin, which prevent release of cytokines from the cell. cNeT are cultured with the following conditions/stimulants:
1. DMSO, Brefeldin A and Monensin as a negative control to show background cytokine production.
2. Staphylococcus enterotoxin B (SEB) as a positive control
3. Long peptide masterpool and Short peptide masterpool corresponding to patient-specific clonal neoantigens reconstituted in DMSO and diluted in water resulting in a final concentration of 0.17 nmols/mL.

Following stimulation, cells are washed and stained with a fixable viability fluorescent dye to enable identification of live cells during analysis. Cells are subsequently fixed, permeabilised, and incubated with fluorescent antibodies specific for the cell surface identification markers CD3, CD4 and CD8 to identify T cells and T cells subsets, and for the intracellular cytokines IFNγ and TNFα to identify T cell function in response to stimulus. Flow cytometry (BD FACSLyric or equivalent) is used to acquire a target of 20,000 live CD3+ cells and data is analysed using the acquisition software FACSuite to identify live CD3+ cells and to calculate total cytokine production. Analysis of cytokine production includes both single (IFN-γ or TNF-α) and dual cytokine-producing cells (IFN-γ and TNF-α). Each condition is run in duplicate and the mean of the duplicates is calculated.

### Calculation of Cell Dose

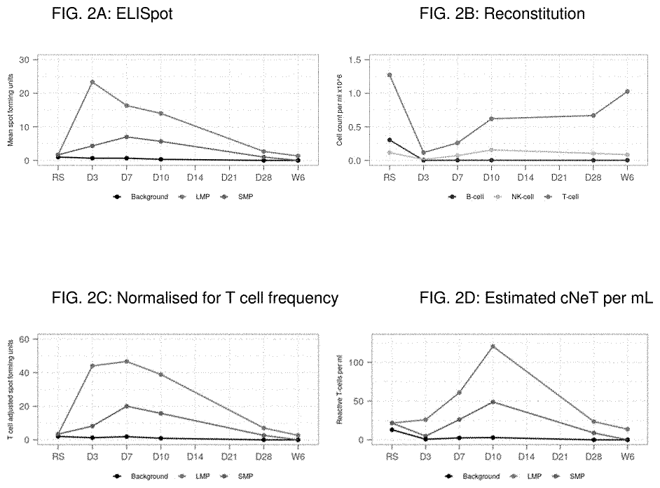

The percentage of CD3+ T cells that respond to stimulus in the intracellular cytokine assay can be used to calculate cell dose. The percentage of CD3+ T cells with IFNγ, TNFα, or dual expression is defined by gating positive cells during analysis of the FACSuite data. This percentage is calculated for the response to short peptide pool, long peptide pool and SEB superantigen. The percentage of peptide-reactive CD3 from the cNeT product for patient T-05 is shown in FIGS. 2A-2D. The percentage of cytokine-positive cells can be used to apply a threshold for batch release or alternatively, the percentage is multiplied by the total number of viable CD3 cells in the product to calculate the reactive cell dose.

Reactive cell dose=[(viable CD3+cell count/drug substance volume)×drug product volume]×percentage reactivity

The reactivity corresponds to the higher value of percentage IFNγ⁺/TNFα⁻ for the short peptides or the long peptides changes in the expression levels of surface markers or markers of T cell activation may be analysed in response to exposure to said antigen(s). These may include, for example, 4-1BB/CD137 (Uniport ID: Q07011), CD25 (Uniprot ID: P01589), OX40/CD134 (Uniprot ID: P43489), Ki67 (Uniprot ID: P46013), Granzyme B (Uniprot ID: P10144), Perforin (Uniprot ID: P14222), CD107a (Uniprot ID: P11279), LAG-3 (Uniprot ID: P18627), PD-1 (Uniprot ID: Q15116), TIM-3 (Uniprot ID: Q8TDQ0), CTLA4 (Uniprot ID: P16410), CD39 (Uniprot ID: P55772), Fas (Uniprot ID: P25445), FasL (Uniprot ID: P48023), CD40L (Uniprot ID: P29965), KLRG1 (Uniprot ID: Q96E93), GITR (Uniprot ID: Q9Y5U5) and ICOS (Uniprot ID: Q9Y6W8). Markers may be used for determining the frequency of antigen-reactive cells in the product.

Similarly, surface markers or markers of T cell activation may be analysed by any suitable method, for example flow cytometry. The antigen may be as described herein. In one aspect the antigen is a subject-specific antigen. In an alternative aspect the antigen is a known tumour antigen.

This value is multiplied by the viable CD3+ cell count in the drug product to determine the dose of T cells reactive to antigens for administration.

Drug substance definition according to the US FDA: Any substance or mixture of substances intended to be used in the manufacture of a drug (medicinal) product and that, when used in the production of a drug, becomes an active ingredient of the drug product.

Drug product definition according to the US FDA: A finished dosage form (e.g. Tablet, Capsule or solution) that contains a drug substance, generally but not necessarily in association with one or more other ingredient.

### ELISpot Reactivity Assay

PBMCs were isolated from whole blood samples collected using Ficoll-Paque (Merck Life Sciences). On the first day of the assay frozen PBMCs were thawed at 37° C., mixed with complete TexMACS media (Miltenyi Biotec)+1% Penicillin/Streptomycin (Life Technologies) and centrifugated at 450×g for 7 minutes. Cells were resuspended in complete TexMACS media and rested at 37° C., 5% CO₂ for 4-6 hours. After resting, PBMCs were centrifugated at 450×g for 7 minutes and resuspended in complete CTL Test Medium (CTL Europe Gmbh)+1% GlutaMAX (Life Technologies).

Peptides were reconstituted in 100% DMSO (WAK-Chemie Medical Gmbh), diluted 1:5 in water (Life Technologies), before dilution in complete CTL Test Medium.

After resting, 2×10⁵ cells per well were plated in 96-well, pre-coated plate (Human IFN-γ⁻ Single Colour ELISpot kit, CTL Europe Gmbh) which had been previously washed with 200 μL DPBS (Life Technologies) twice. 100 μL of negative control (0.66% DMSO), positive control (2 μg/mL Staphylococcal enterotoxin B, Merch Life Sciences) or peptides for testing were added to each well at resulting in a final concentration of 0.000165 nmol/μL for short and long peptide masterpools. Plates were incubated at 37° C., 5% CO₂ for 12-16 hours. Detection antibodies and developing solution were added as per manufacturer's instructions before reading on CTL ELISpot plate reader (Bio-Sys Gmbh Bioreader 6000-Fγ⁻).

### TBNK Assay

50 μL whole blood sample was stained with Multitest™ 6-colour TBNK reagent (BD Biosciences) according to manufacturer's instructions. Prior to sample acquisition, 1 μL of 1 μg/mL 4′,6-diamidino-2-phenylindole (DAPI, BD Biosciences), a DNA binding dye, was added to whole blood samples immediately before sample analysis. Samples were acquired on BD FACSLyric™ and analysed using BD FACSuite™ software.

### Results

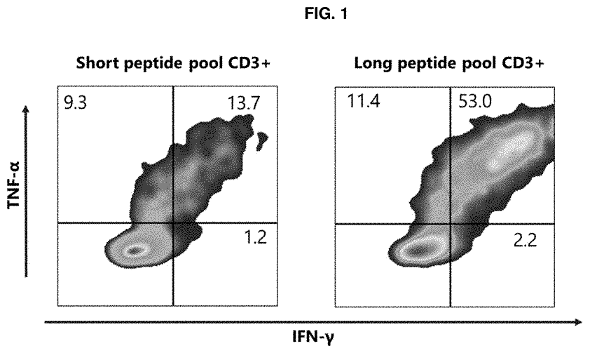

The potency of the manufactured product was measured by intracellular cytokine secretion of IFN-γ and TNF-α using flow cytometry (FIG. 1). This shows, along with the multiple reactivities identified by ELISpot analysis, the presence of single as well as multi-functional cytokine-secreting cNeT. cNeT were tracked pre- and post-dosing in an IFNγ ELISpot assay using the long and short peptide pools that incorporate the identified clonal mutations (FIG. 2A). Adjusting for the impact of immune system reconstitution observed in TBNK assay (FIG. 2B) allows normalisation for T cell frequency in the ELISpot assay (FIG. 2C) and provides an estimate of the cNeT count/mL in peripheral circulation (FIG. 2D). This shows expansion and detection of cNeT post dosing and provides an estimate of the quantity of reactive T cells in circulation. This confirms that the use of antigen in a batch release assay is a relevant method for characterising the active component of a product that can then be detected post-dosing.

The inventors have found that the reactivity and dose of a cell product can be assessed using patient-specific peptides. Surprisingly, the specific reactivity of the T cells can be sufficiently shown using the peptides described herein without the need of antigen presenting cells (APCs) (FIG. 1). While using APCs may provide a higher response, the T-to-T cell assay as described herein, is advantageous as it does not require additional cell cultures or the need to generate other cells lines (e.g. such as dendritic cells or B cells). The assay described herein therefore provides a more easily validated and scalable assay than an assay that requires additional cell cultures or the generation of other cell lines.

### Identification and Production of Antigen

Blood and tumour samples were obtained from each patient and whole exome sequencing (WES) was carried out. The proprietary PELEUS™ bioinformatics platform was used to carry out the following steps:

(i) identify patient-specific somatic mutations (including single nucleotide variants (SNVs), multiple nucleotide variants (MNVs) and insertions/deletions (indels)) by comparing DNA sequence data from the germline (blood) sample and the matched tumour samples to each other and to a reference genome;
(ii) identify a set of mutations that are likely to be clonal in view of the sequence data from the patient using a Bayesian approach (see e.g. McGranahan et al., Science Vol 135:6280, p. 1463-1469; Roth et al., Nat Methods. 2014 April; 11(4): 396-398); and
(iii) design a set of peptides comprising the set of somatic mutations identified as likely to be clonal and non-silent.

The resulting set of candidate antigenic peptides was manufactured using standard peptide synthesis methods.

### Example 3

Surface Activation Marker Profile of cNeT Product Upon Restimulation

1,200,000 cNeT cells were cultured with DMSO (negative control), SEB (positive control), or patient specific clonal neoantigen peptides (0.033 nmol) for 44-52 hours.

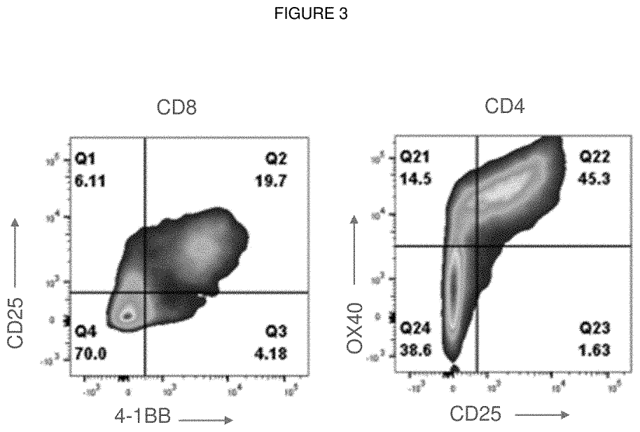

Cell pellets were collected and CD25, 4-1BB and OX40 surface molecules were stained using fluorescent-dye conjugated antibodies and measured by flow cytometry. Data were analysed by Flowjo software and expression was shown for CD8 (left) and CD4 (right) T cell subsets in smoothed pseudocolor plots (FIG. 3). The data shows that stimulation of cNeT with specific peptides induces expression of (i) CD25 and 4-1BB on CD8 T cells and (ii) CD25 and OX40 on CD4 T cells.

### Example 4

Percentage of cNet Product Secreting Cytokines

B cells were isolated from matched patient PBMC and expanded using IL4, CpG ODN 2006, anti-immunoglobulin and CD40L. Mature dendritic cells were generated by standard methods for differentiation from monocytes, followed by maturation. 200,000 cNeT cells were cultured with either B cells (a) or mature DC (b) pulsed with a pool of long peptides (0.033 nmol) for 16-17 hours in the presence of protein transport inhibitor brefeldin A and monensin to stop cytokine secretion.

Cells were intracellularly stained for total IFNγ and TNFα expression using fluorescent-dye conjugated antibodies and measured by flow cytometry. Background level of expression detected from DMSO negative control was subtracted. Percent of expression in CD4 T cell subset is listed in FIGS. 4A and 4B.

The data shows that cytokine expression by CD4 T cells is comparable between B cells and mDC as antigen presenting cells.

### Example 5

Mulitplexed Cytokine Secretion by cNeT Product

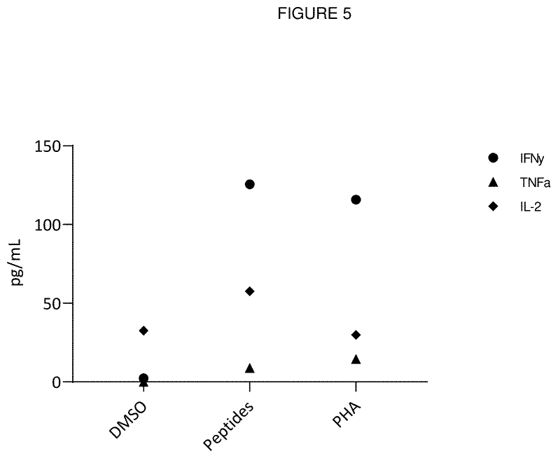

100,000 cNeT cells were cultured with DMSO (negative control), PHA (positive control), or patient specific clonal neoantigen peptides (0.033 nmol) for 20-24 hours. Supernatants were collected and multiplexed quantification of cytokines was performed by cytometric bead array (CBA) assay using flow cytometry. Concentrations were determined using standard curve (FIG. 5).

The data shows that IFNγ in response to the peptides was detected. Low level of TNFα and IL-2 was also measurable above DMSO background.

### Example 6

Cytokine Secretion by cNeT Product.

1.2 million cNeT cells were cultured with DMSO (negative control), SEB (positive control), or patient specific clonal neoantigen peptides (0.033 nmol) for 44-52 hours.

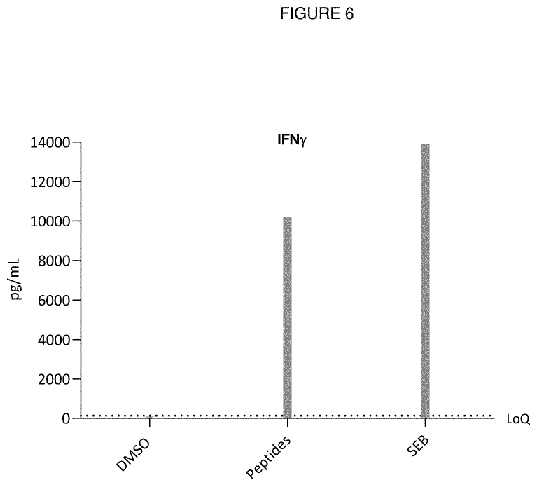

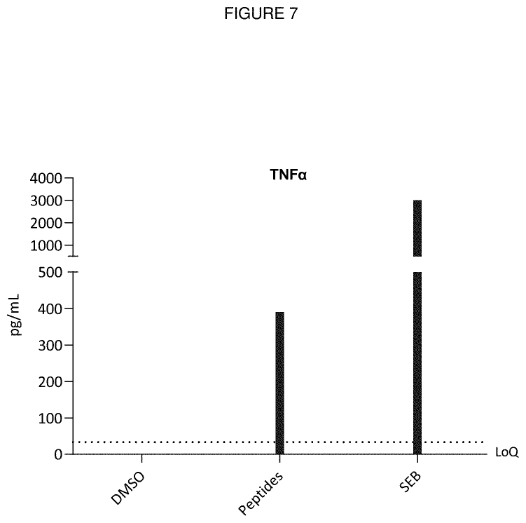

Supernatants were collected and IFNγ (FIG. 6) and TNFα (FIG. 7) were measured and quantified by cytometric bead array (CBA) assay using flow cytometry. Data in FIGS. 6 and 7 show mean of duplicate wells and positive control from a single well.

### Example 7

Peptide Reactivity in Blood cNeTs.

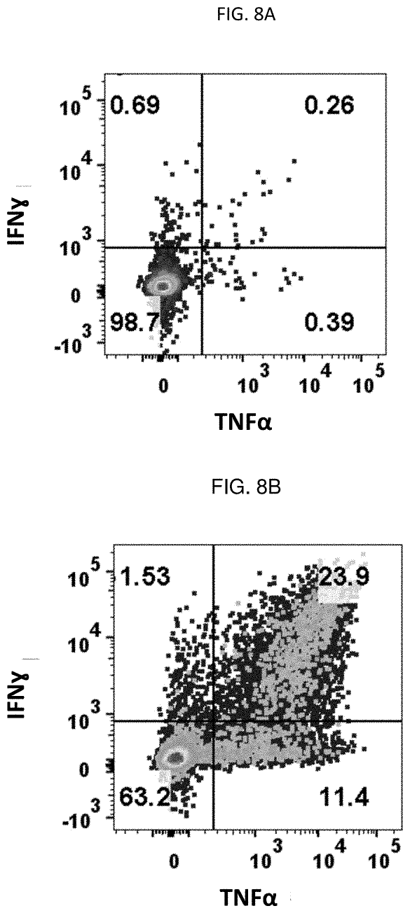

The potency of the blood derived cNeT, retrieved from consented cancer patients, was measured by intracellular cytokine secretion of IFN-γ and TNF-α using flow cytometry (FIGS. 8A and 8B). CD3 reactivity in a “recall” assay was performed overnight and data was generated from memory T cells. Both plots A and B were gated from Live⁺CD3⁺ cells.

This data shows the presence of single as well as multi-functional cytokine-secreting cNeT (B) compared to that of the unstimulated cells (brefeldin and monensin only), the negative control (A).

### Example 8

Response of cNeT Product Upon Restimulation

200,000 cNeT cells were cultured with either DMSO (negative control), or patient specific clonal neoantigen peptides for 16 hours.

Cell pellets were collected, and fluorescent anti-surface receptor antibodies: 4-1 BB and CD107a; CD40L and CD25 were subsequently used to quantify the frequency of responding cells by flow cytometry. Data were analysed using FlowJo software and expression was shown for CD3+ CD56− T cells.

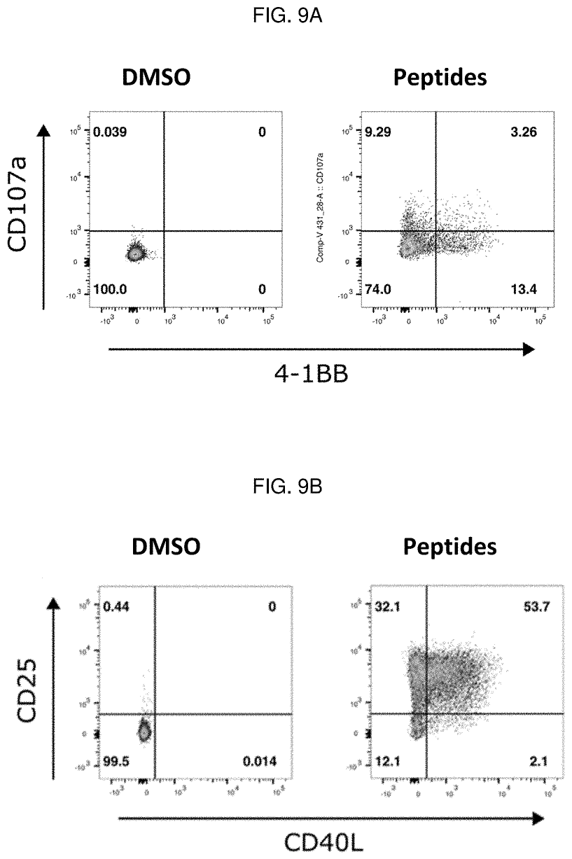

The data shows that stimulation of cNeT with specific peptides induces a polyfunctional response, with different percentages of cells responding by expressing the degranulation marker CD107a or surface effector molecules 4-1BB, CD40L or CD25 (FIGS. 9A and B). All the presented markers can be used to evaluate the frequency of responding cells in the product.

What is claimed is:

1. A method of treating a subject that has been diagnosed as having cancer, the method comprising:
a) providing a sample of a pharmaceutical product comprising T cells;
b) analysing reactivity of the T cells of the sample to an assay antigen;
c) determining that said T cells meet a predetermined threshold for reactivity to the assay antigen; and
d) if the T cells meet the predetermined threshold, administering the pharmaceutical product to the subject,
wherein the pharmaceutical product comprises tumour infiltrating lymphocytes (TILs) isolated from a tumour sample from the subject.

2. The method according to claim 1, wherein the assay antigen is a tumour associated antigen (TAA) or tumour specific antigen (TSA).

3. The method according to claim 2, wherein the assay antigen is a neoantigen of the subject.

4. The method according to claim 3, wherein the assay antigen is a clonal neoantigen of the subject.

5. The method according to claim 1, wherein step b) is carried out in the absence of antigen presenting cells (APCs).

6. The method according to claim 1, wherein step b) is carried out in the absence of any other cell types.

7. The method according to claim 6, wherein the T cells present said assay antigen to other T cells (T-to-T cell assay).

8. The method according to claim 7, wherein the reactivity of the T cells to the assay antigen is analysed by measuring expression level of at least one cytokine and/or measuring expression level of at least one T cell marker or T cell surface marker.

9. The method according to claim 8, wherein the at least one cytokine comprises IFN-γ and/or TNF-α.

10. The method according to claim 8, wherein the at least one T cell marker or T cell surface marker comprises 4-1 BB, CD25, OX40, Ki67, Granzyme B, Perforin, CD107a, LAG-3, PD-1, TIM-3, CTLA4, CD39, Fas, FasL, CD40L, KLRG1, GITR and ICOS.

11. The method according to claim 8, wherein T cell reactivity is determined by flow cytometry, immunoassay, immunoassay, ELISpot and/or TCR sequencing.

12. The method according to claim 1, wherein said assay antigen has been identified prior to or after T cell expansion.

13. The method according to claim 1, wherein said pharmaceutical product comprises T cells that have been expanded in vitro.

14. The method according to claim 12, wherein said T cells of the pharmaceutical product have been expanded in the presence of an expansion antigen prior to providing the sample for the analysing of reactivity.

15. The method according to claim 14, wherein said assay antigen has been identified as a neoantigen of the subject prior to or after T cell expansion.

16. The method according to claim 13, wherein the same antigen is used as both the assay antigen and the expansion antigen.

17. The method according to claim 13, wherein different antigens are used as the assay antigen and the expansion antigen.

18. The method according to claim 1, wherein the threshold of reactivity is met when the number of reactive T cells is at least 1×10⁵ reactive cells.

19. The method according to claim 1, wherein said pharmaceutical product comprises engineered T cells.

20. The method according to claim 1, wherein the cancer is melanoma or non-small cell lung cancer (NSCLC).
